(19) United States
(12) Patent Application Publication
(10) Pub. No.: US 2014/0180549 A1
(43) Pub. Date: Jun. 26, 2014

(54) AUTOMATED MACHINE FOR SELECTIVE IN SITU MANIPULATION OF PLANTS

(71) Applicants: Mark C. Siemens, Yuma, AZ (US); Ronald R. Gayler, Yuma, AZ (US); Kurt D. Nolte, Yuma, AZ (US); Ryan Herbon, Silver City, NM (US)

(72) Inventors: Mark C. Siemens, Yuma, AZ (US); Ronald R. Gayler, Yuma, AZ (US); Kurt D. Nolte, Yuma, AZ (US); Ryan Herbon, Silver City, NM (US)

(73) Assignee: The Arizona Board of Regents on Behalf of the University of Arizona, Tucson, AZ (US)

(21) Appl. No.: 13/978,378

(22) PCT Filed: Dec. 14, 2011
(86) PCT No.: PCT/US11/64957

§ 371 Date: Feb. 5, 2014

Related U.S. Application Data
(60) Provisional application No. 61/460,799, filed on Jan. 7, 2011.
(60) Provisional application No. 61/552,728, filed on Oct. 28, 2011.

(51) Int. Cl. A01G 1/00; G01N 33/00; A01C 15/00; A01B 39/18; A01G 25/09; A01G 3/00

(52) U.S. Cl. CPC A01G 1/00; A01G 25/09; A01G 3/00; A01C 15/00; A01B 39/18; G01N 33/0098
USPC 701/50; 348/89

(57) ABSTRACT

Disclosed herein are systems for identifying a plant in a field with pixel-level precision and taking an action on the identified plant, also with pixel level precision. The disclosed systems can include multiple implements, enabling the performance of multiple tasks on an identified plant or associated area of soil. Methods of using the described systems for automated thinning, weeding and spot spraying crops are also disclosed.

### CROSS REFERENCE TO RELATED APPLICATION

This application claims priority to and the benefit of U.S. Provisional Application 61/460,799, filed on Jan. 7, 2011 and U.S. Provisional Application 61/552,728, filed on Oct. 28, 2011, both of which are incorporated herein by reference in their entirety.

### ACKNOWLEDGMENT OF GOVERNMENT SUPPORT

This invention was made in part with government support from the United States Department of Agriculture under the Arizona Department of Agriculture Specialty Crop Block Grant Program—SCBGP Grant No. SCBGP-FB09-19. The government has certain rights in the invention.

### FIELD

This disclosure relates to, inter alia, systems and methods for in-field real-time identification of a plant, and performance of an action on the identified plant.

### BACKGROUND

Agriculture is a multi-billion dollar global industry. Among the challenges to agricultural economic viability are escalating labor costs and a shortage of readily available labor due to increasingly stringent immigration policies in many countries. The cost and shortage of available labor is of particular concern for producers of crops requiring intensive manual attention.

For many crops, readily-available low-cost manual labor is needed for multiple manual operations throughout the growing season. Plant thinning is an example of a particularly labor-intensive operation. Because many crops are sown in greater numbers than the desired final plant population to ensure adequate stand establishment, plant thinning is necessary to prevent overutilization of available resources, to ensure optimum crop size and quality, and to facilitate later harvesting. Currently, this is most-commonly accomplished by a crew of workers using hand hoes or other suitable tools.

Automated devices for manual operations such as plant thinning, weeding, and spot spraying can be grouped into two general categories: fixed-interval thinners and selective thinners. Fixed-interval thinners typically use an oscillating hoe or a rotating blade to remove “blocks” of plants at fixed intervals along a crop row length. Selective thinners utilize sensors to detect plants and then, depending on plant location, selectively remove unwanted plants.

A major drawback of fixed-interval thinners is that that they have no means for determining which plants to leave alone and which to remove. Thus, when plant spacing is irregular, a fixed-interval thinner is just as likely to remove desired plants as unwanted plants, and leave large spaces in the crop row. Although sensor-based systems overcome some of these problems, the sensor-based systems developed to date remain imprecise and/or too slow to be commercially viable. The majority of prior sensor-based systems are “area” or “spot” based systems that analyze and treat unitary areas of a predetermined, constant dimension in response to a signal. Such systems are inherently limited and do not have the precision and flexibility needed for economical automated crop handling in situ. Operation precision is a particular concern when thinning closely seeded crops, such as lettuce, that are easily damaged by the excessive plant-bed disturbance and soil-throw associated with many automated plant thinners. For example, U.S. Patent Publication No. US 2011/0211733 describes a sensor-based plant thinner system, but provides no guidance for identifying plants in the field with the precision, speed, and flexibility needed for economical automated crop handling. Thus, a continuing need exists for precise, automated devices for performing otherwise manual operations on crops in a field.

### SUMMARY

Disclosed herein are systems for identifying a plant in a field with pixel-level accuracy and taking an action on the identified plant in situ, also with pixel-level accuracy. The disclosed systems can include one or more implements controlled by a controller responsive to the obtained data, thereby enabling the performance of one or more tasks on certain identified plants or associated areas of soil.

In particular embodiments the systems include: (1) a support movable in a trajectory along an array of plants; (2) an image sensor, including a camera, which is mounted to or relative to the support, and which is capable of producing real-time images on an electronic image-capture device containing an array of pixels; (3) a distance-measuring device that produces, in real time, data regarding position of the support in the trajectory relative to a positional reference; and (4) a first controller connected to the image sensor and to the distance-measuring device, which is programmed or otherwise configured: (a) from the data obtained from the distance-measuring device and at selected discrete distances in the trajectory from the reference, to generate an activate signal triggering the image sensor to obtain an image of a respective region of interest (ROI) of the array situated at the respective selected distance from the reference, (b) to receive pixel-level image data of the ROI image from the image sensor, (c) at selected pixels of the image (e.g., at each pixel of the image), to determine whether light received at the pixel is indicative of plant versus non-plant, (d) to determine a data distribution of plant-indicating pixels and non-plant-indicating pixels as a function of distance in the ROI and hence relative to the reference, and (e) in the distribution, determine respective positions of leading and trailing edges of plant-indicating pixels and correlating these positions with desired action or non-action to be taken with respect to selected plants in the ROI. The described systems can include an optional user interface for programming the controller and displaying data of the plant-indicating and non-plant-indicating pixels in the ROI. The user interface can be mounted to the support so as to be available at any time to an operator, or can be disconnectable and removable to protect it from damage and contamination that may be encountered in the field. The user interface desirably includes a display and a keyboard or other user-manipulatable controls as required.

In particular embodiments of the described systems, the first controller is further programmed to produce, with respect to a plant in the ROI determined to be at a position correlated with action, at least one implement-actuation command to take at least one desired action on, or relative to, the plant. In other embodiments, the systems include a second controller, which is programmed to produce, with respect to a plant in the ROI determined to be at a position correlated with action, at least one implement-actuation command to take at least one desired action on, or relative to, the plant. In such embodiments, the user interface can also be used to program the first or if present, the second controller.

In particular embodiments, the systems include at least one implement, such as one or more spray nozzles or blades, connected to either the first controller or second controller (if present), wherein the implement receives the actuation command from the respective controller and executes the corresponding action, for example to manipulate a plant or region of soil associated with the plant. In other embodiments, the systems include multiple implements, each of which receives a different implement-actuation command at the appropriate moment in time from the respective controller. Upon receiving a command, each implement executes the desired action, for example, to manipulate a plant or region of soil associated with the plant. For example, the implements can include one or more spray nozzles positioned on the support to direct, in real time as commanded by the respective controller, a substance at or near a selected plant. An exemplary substance in this regard is a liquid, such as an acid, used for killing the selected plant, a plant nutrient for enhancing growth of the selected plant, or water for irrigating the selected plant. Generally, in the various embodiments having spray nozzles, the spray nozzle(s) is made of material resistant to the substance discharged by the nozzle.

In some embodiments, the support is pulled or pushed along the trajectory by a motile device such as a tractor or the like. In other embodiments, the support is self-motile and is a tractor or other motor vehicle. It is also possible that the support be pulled or pushed by stationary devices such as a motor with a pulley and cable connected to the support.

The distance-measuring device is any device suitable for measuring the position of the support, whether it is stationary or moving in a field. By way of example, in various respective embodiments, the distance-measuring device includes a rotary encoder, a linear encoder, a radar device, and/or a global positioning system.

Additionally disclosed herein are various agricultural systems that include support means movable in a trajectory along an array of plants. The system includes means, coupled to the support means, comprising a camera, for producing pixelated electronic images of respective portions of the array, wherein each image consists of an array of pixels. Hence, a “camera” as used herein comprises any camera known in the art that will capture an image as an array of pixels. Exemplary cameras include trigger-activated cameras that are capable of receiving an electrical signal that controls shutter activation. Other exemplary cameras are digital still and/or video cameras that have any type of image sensor known in the art, such as charged coupled device (CCD) and complementary metal-oxide-semiconductor (CMOS) sensors. The system includes means for measuring distance of the support means in the trajectory relative to a positional reference. Exemplary means include, but are not limited to, one or more optical shaft encoders, linear (tape) encoders, magnetic encoders responding to a series of magnets extending along the array above or below ground, mechanical odometers, GPS systems, laser range-finders, radio-based distance-measuring devices (radar), and the like. The system includes means for actuating the camera to take respective images of respective regions of interest (ROIs) of the plant array along the trajectory at respective selected distances from the reference. An exemplary means in this regard is a controller, processor, or computer to which the camera is electronically connected. The system includes means for determining, in each image, whether light received at selected pixels (e.g., at each pixel thereof) is indicative of plant versus non-plant. An exemplary means in this regard is a controller, processor, or computer. The system includes means for determining, in each image, respective positions of leading and trailing edges of plant-indicating pixels and for correlating these positions, at pixel-level resolution, with desired action or non-action to be taken with respect to selected plants in the ROI. An exemplary means in this regard is a controller, processor, or computer. The system further includes optionally removable means for programming the system and optionally removable means for displaying the electronic images and information about the plant versus non-plant pixels in the ROI.

In particular embodiments configured to take action with respect to certain plants detected by the system, the systems further include implement means mounted to the support means; and means for actuating the implement means to take action with respect to a plant in the ROI determined to be at a position correlated with the action. Such implement means can be one or more of a spray nozzle or blade. An exemplary means for actuating the implement means is a controller or portion thereof that is responsive to data regarding selected plants and that is configured to produce or implement actuation commands receivable by the implement means to actuate the implement means at the appropriate time.

Additionally described herein are methods for manipulating plants in situ while moving at least one implement in a trajectory along an array of plants. The methods include determining the position of the implement in real time relative to a positional reference. The methods include obtaining in real time, a series of pixelated images of respective portions of the array located in respective regions of interest (ROI) situated at discrete respective distances from the reference. The methods include determining, in each image, whether respective image light received at the pixels is indicative of plant versus non-plant; and determining respective leading and trailing edges of plant-indicating pixels and correlating these positions, at pixel-level resolution, with desired action or non-action to be taken with respect to selected plants in the respective ROI. The methods further include actuating the implement to take action with respect to a plant in the ROI determined to be at a position correlated with the action.

In particular embodiments, the implement includes a nozzle (e.g., a nozzle made of material resistant to the substance discharged by the nozzle) or a blade. In other embodiments, the action is plant thinning, weeding, spot spraying, watering, or fertilizing.

In other embodiments, the methods include at least one additional action with respect to the plant in the ROI, such as additional plant thinning, weeding, spot spraying, watering, or fertilizing.

The foregoing and other objects, features, and advantages of the invention will become more apparent from the following detailed description, which proceeds with reference to the accompanying figures.

### BRIEF DESCRIPTION OF THE DRAWINGS

FIG. 1 is a schematic diagram showing, in block form, general features of an exemplary embodiment of an automated machine for in-field plant identification and selective treatment of identified plants.

FIG. 2 is a rear view of a first representative embodiment of an automated machine for in-field plant identification and selective treatment of identified plants.

FIG. 3 is a side view of a second representative embodiment of an automated machine for in-field plant identification and selective treatment of identified plants.

FIG. 4A is a flow chart showing an overview of the processes carried out by the firmware and software of an embodiment of the automated system for in-field identification and selective treatment of identified plants.

FIG. 4B is a flow chart illustrating the processes carried out by the firmware of an embodiment of the automated system for in-field identification and selective treatment of identified plants.

FIG. 4C is a flow chart illustrating an overview of an embodiment of the processes carried out by “slave” controller software for communication between the “master” controller and the firmware.

FIG. 4D is a subprocess flow chart of the “slave” controller process diagram, illustrating the image processing of an embodiment of the described automated systems.

FIG. 4E is a subprocess flow chart of the image processing process diagram, illustrating the plant location identification processes of an embodiment of the described automated systems.

FIG. 4F is a subprocess flow chart of the image processing process diagram, illustrating the process for determining which plants to keep in an embodiment of the described automated systems.

FIG. 5 is a flow chart showing an embodiment of a method for manipulating plants in situ.

FIG. 6 is a drawing showing alternating strips of colored dye solution sprayed on a crop row as used for calibrating distance measurements obtained using an optical shaft encoder distance measurement.

FIG. 7 is a drawing showing a calibration board of alternating white and black strips as used by certain embodiments for calibrating pixel size and real-world distances.

FIG. 8 depicts an exemplary user-interface screen displaying icons relevant to calibration of an embodiment of the vision imaging system in relation to real-world distances. The captured image in the lower right of the figure is of a calibration board of alternating black and white strips of known width. Best-fit cubic-regression analysis of image data obtained using the calibration board is used to determine a prediction equation for estimating the number of pixels per real-world inch. The plot in the upper right corner of the figure depicts an example of calibration accuracy.

FIG. 9 depicts an exemplary user-interface screen showing user input fields that can be used for adjustment of selected pixel hue, saturation, and luminance values, for example. Also shown are input fields for adjusting the size of the region of interest (ROI) for analysis, and two exemplary ROIs.

FIG. 10 depicts an exemplary user-interface screen showing an exemplary analysis of a captured image. The upper panel is a close-up view of a portion of an analyzed ROI. ROI pixels determined to be plant parts are outlined and depicted in white. Leading and trailing edges of the three plants in the ROI are indicated by the boxes. The lower panel shows a frequency distribution of the plant pixels across all rows in the ROI plotted versus distance relative to each one-pixel width column.

FIG. 11 depicts an exemplary user-interface screen that can be used for adjusting operating parameters of the system as applicable to automated plant operations performed by the system. Adjustable parameters include but are not limited to, one or more of desired plant spacing, minimum plant spacing, leading-edge buffer distance from plant edges, trailing-edge buffer distances from plant edges, minimum plant length, array filter size, and minimum noise level.

FIG. 12A depicts a close-up of a portion of a user-interface screen showing a ROI analyzed for plant location and selective treatment. Plants A and B and the following user-defined distance parameters are indicated: plant length (L), trailing edge buffer distances from plant edges (TE_{bd}), leading-edge buffer distances from plant edges (LE_{bd}), minimum plant-spacing distance (Dₘᵢₙ), desired plant-spacing distance (D_{desired}), distance to next selected plant (Dₚₗₐₙₜ), and distance treated (D_{treated})

FIG. 12B is a close-up of a portion of a user-interface screen showing a ROI analyzed for plant location and selective treatment in a “two-drop” planting scheme. Plant length (L), leading-edge buffer distance from plant edges (LE_{bd}), and distance treated (D_{treated}) are indicated.

FIG. 13 depicts results of a performance trial of an embodiment of the system as used for automated plant identification and treatment with a pressurized spray. Two rows of lettuce seedlings are presented on each crop bed. The rows in the right-hand crop bed were not thinned. The far left row was machine thinned. The directly adjacent right-hand row was hand-thinned, thus comparing and illustrating the fidelity of machine action and hand action in the same field.

### DETAILED DESCRIPTION

Many crops depend on multiple manual operations during a growing season. In view of the increasing shortage of available manual labor, automation of otherwise manually accomplished tasks is rapidly becoming a requirement for agricultural economic viability. Automated systems have been previously developed for identifying and performing action on plants. However, the automated systems developed to date cannot operate with the precision and/or speed necessary for practical treatment of a region of interest (ROI) near crop plants (i.e., thinning closely spaced seedlings, inter-row and intra-row weeding). For example, the automated system described in U.S. Patent Publication No. US 2011/0211733 provides no guidance pertaining to how the system identifies plants in a field, let alone with the precision required for automated plant treatment. The system described in this reference also provides no guidance for precisely distinguishing the desired crop plants from either unwanted crop plants or weeds. In contrast, the systems described herein identify plant boundaries with high resolution (pixel-level precision in many instances), and therefore can take action on selected plant or surrounding areas with very high precision.

Although the systems and methods are described herein with respect to crop plants, and most particularly lettuce crops, the systems and methods described herein can be used for any type of plant, including any type of crop plant that requires one or more specific operations during a growing season, including thinning, weeding, and spot spraying.

Unless otherwise explained, all technical and scientific terms used herein have the same meaning as commonly understood by one of ordinary skill in the art to which this disclosure belongs. The singular terms “a,” “an,” and “the” include plural referents unless the context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The term “comprises” means “includes.” All publications, patent applications, patents, and other references mentioned herein are incorporated by reference in their entirety for all purposes. In case of conflict, the present specification, including explanations of terms, will control. In addition, the materials, methods, and examples are illustrative only and not intended to be limiting.

### Overview of Several Embodiments

Disclosed herein are systems for in-field (in situ), real-time, high-resolution identification of plants. The identifications can be the basis upon which any of various selected operations can be executed by the systems against or with respect to selected plants. The systems include the following components: (1) a movable support that can be moved along an array of plants; (2) an image sensor that includes a camera mounted to the support; (3) a distance-measuring device that produces, in real time, data regarding the distance moved by the support along the array of plants; (4) a controller connected to the camera and the distance-measuring device, which coordinates the position of the support, activates the camera at specified distances, and processes the captured image to identify plants at particular locations in the array; and (5) a display and user-interface (optionally removable) through which the controller can be programmed and data of plant location can be output or displayed.

In particular embodiments, the described systems are adapted for performance of an action on an identified plant. In such embodiments, the described systems additionally include at least one implement that is connected to the controller, and which is activated by the controller to take an action on or with respect to a selected plant, based at least in part on the position of the plant as determined by the system.

### Movable Support

The components of the systems disclosed herein are mounted on, within, or relative to a movable support, such as a motile vehicle or analogous device suitable for a particular agricultural situation. In particular embodiments, the motile vehicle can be a tractor. In particular examples, the components of the system is mounted to a trailer, a cart, or the like, wherein the trailer or cart is coupled to and pulled or pushed by a motile vehicle such as a tractor. The vehicle need not be powered by an internal combustion engine; it alternatively could be electrically powered, for example. The vehicle need not be self-powered at all; it could be pulled by a cable, for example, across a field.

In particular embodiments, the support is a frame composed of metal or other suitable material that can attach to any vehicle known in the art, such as a tractor, via any suitable attachment means, such as a three-point hitch. Other exemplary attachment means include a drawbar hitch.

In particular examples, the support includes means, such as guide cones, to keep the system centered on the desired crop row(s) and gauge wheels for maintaining height of the system relative to soil level in the row(s). In other embodiments the support is configured to maintain a constant height as the support moves along a plant bed.

It will be understood by those skilled in the art that the systems disclosed herein can be adapted for simultaneous use on multiple plant rows by enlarging and/or replicating the system components depicted and described herein. For example by attaching multiple supports or by using larger supports to which multiple cameras, controllers, and treatment means (e.g. nozzle assemblies, blades, and the like) may be attached.

### Image Sensor

The image sensor is a so called “machine-vision” imaging system that comprises an electronic image-capturing device. The image-capturing device may be part of a digital camera, for example. Various embodiments of the image sensor comprise a trigger-activated camera that is capable of receiving an electrical signal that controls shutter activation to capture a pixelated image of a region of interest (ROI). The shutter-activation signal is delivered to the camera whenever the support moves a preset distance, as measured by the distance-measuring device. The camera need not operate continuously (although it potentially could). Rather, in particular embodiments, the shutter-activation signal is delivered intermittently to the camera (after the support has moved a designated distance) so that the camera obtains discrete images ROI-by-ROI. The obtained ROIs can, but need not, overlap each other, depending on the distance traveled by the support between shutter-activation signals.

The digital camera can have any type of image-capturing device known in the art, such as a charged coupled device (CCD) or a complementary metal-oxide-semiconductor (CMOS) sensor. Any camera known in the art that will capture an image as an array of pixels may be used in the systems and methods described herein.

In most embodiments, and for most uses, the image sensor is sensitive to one or more wavelengths of visible light; but under other circumstances it may alternatively or additionally be sensitive to one or more other wavelengths, such as of infrared (IR) light. In particular embodiments, in which the image sensor is sensitive to visible light, the imaging camera captures digital images in standard red (R), green (G), blue (B) format so that each light-stimulated pixel in the captured image has associated R, G, and B values. In particular embodiments, the controller is programmed to convert these values to corresponding hue (H), saturation (S), and luminance (L) values by methods known in the art. In other embodiments, the camera itself has the capability to convert RGB values to HSL values before transmitting pixel information to the controller. In still other embodiments, the camera captures images in monochrome/black and white so that each pixel is initially captured with an associated HSL value.

The camera may be mounted to or situated relative to the support by any means known in the art. In particular examples, the camera is exposed to the environment, and receives light from natural sources, notably sunlight. In other examples, one or more light sources can be associated with the camera to provide or augment the imaging light. In such examples, the camera can be situated in a partial enclosure in or to which the light source(s) can be optionally affixed for consistent lighting of the surface of a field. In some examples, both the camera and the enclosure are mounted to the support. In other examples, the enclosure is mounted to the support and the camera is mounted to the enclosure. In still further examples, the camera is mounted to the support and the enclosure is attached to the camera. In yet further examples, the camera and light source are in separate enclosures that are open exposed to the ground containing the array of plants.

### Distance-Measuring Device

The distance-measuring device is also attached to or situated relative to the support and provides a means for determining “real-world” physical locations of each pixel in each captured image. The distance-measuring device also provides the controller with information on the distance travelled by the support and hence by the image sensor. Based on this information, the controller determines whether to send a shutter-activate signal to the camera.

The distance-measuring device can comprise any means known in the art for measuring movement of the support, including one or more optical shaft encoders, linear (tape) encoders, magnetic encoders responding to a series of magnets extending along the array above or below ground, GPS systems, laser range finders, radio-based distance-measuring devices (radar), and the like.

In a particular embodiment, the distance-measuring device is a digital or analog encoder or analogous device, such as a rotary or shaft encoder. The encoder accurately counts pulses associated with rotation of a ground-following wheel (for example, to a resolution of 1000 pulses per revolution) over the ground in the direction of movement of the system. Such a wheel is mounted to the support, contacts the ground, and rotates whenever the support is moving relative to the ground. In particular embodiments the rotary encoder is connected to and measures rotations of a wheel of the movable support. In other embodiments, wherein the support is pushed or pulled through a plant array, the rotary encoder can be connected to and measure rotations of a wheel of the motile vehicle to which the support is attached.

In still other embodiments, accuracy of distance measurements can be increased through use of multiple shaft encoders. Distance-measurement accuracy can also be improved through use of higher-resolution encoders (i.e., that detect additional pulses per wheel rotation, such as 2000, 3000, 4000, 5000, 6000, or more pulses per rotation) and/or increasing measured angular rotation per pulse for a given distance traveled. Methods of increasing measured angular rotation are known in the art and include methods such as reducing the ground-following wheel diameter or measuring the rotation of a shaft that is not directly attached to the ground driven wheel, and whose rotational speed has been reduced.

In other examples, distance-measurement accuracy can be improved by user-adjusted calibration of the distance-measuring device. Methods of calibrating an optical encoder are known in the art, for example, any of various standard techniques of counting the number of encoder pulses over a given travel distance and inputting the ratio of these two numbers (e.g., number of pulses/inch).

In other embodiments, the distance-measuring device is a GPS device capable of accurately detecting the position and distance traveled of the support. In still further embodiments, the distance-measuring device combines a GPS device with one or more additional devices capable of detecting support movement (e.g. an encoder).

### Controller and User Interface

Systems as described herein comprise one or more controllers that control at least certain aspects of the operation of the described systems. A “controller” is usually a computer processor that is programmed to execute particular operations of the system. Alternatively, the controller can be for example, “hard wired” to execute predetermined operations in a predetermined way. In most embodiments, the controller is programmed or otherwise is configured (e.g., by software and/or firmware) to execute at least the following functions:

(a) from the data obtained from the distance-measuring device and at selected discrete distances from a reference location, signal the camera to obtain an image of a region of interest (ROI), such as an image of an array of plants in a particular region of the field in which the system is operating;

(b) receive pixelated image data obtained by the camera at the ROI;

(c) at selected pixels of the image (e.g., at each pixel of the image), determine whether light received at the pixel is indicative of plant versus non-plant;

(d) determine a data distribution of at least some of the plant-indicating pixels and non-plant-indicating pixels as a function of distance in the ROI and hence relative to the reference; and

(e) in the data distribution, determine respective positions of leading and trailing edges of plant-indicating pixels, and correlate these positions with desired action or non-action to be taken with respect to selected plants in the ROI.

In particular embodiments, the controller analyzes groups of pixels in the ROI and correlates action or non-action in relation to the group. In other embodiments, the controller analyzes the ROI pixel-by-pixel and thus is able to correlate action on with single-pixel precision.

In particular embodiments, the controller is further programmed to send an implement-actuation command signal to at least one implement of the system to execute a respective action on selected plants in the ROI.

In particular embodiments, the described systems have multiple controllers for carrying out specified actions of the described systems. In certain embodiments the multiple controllers are configured to be in a “master” and “slave” configuration wherein the “master” controller sends program operation settings to the “slave” controller, which carries out the functions of the automated systems. By way of example, in a particular embodiment with two controllers, the first controller is programmed to receive and analyze data from the distance-measuring device, send shutter-activation signals to the camera, receive digitized image information, analyze the digitized images, make control decisions based on the analysis of the images, and send electronic control decision outputs to the second controller. The second controller receives an input, namely decision outputs from the first controller. Based on this input and on input from other sensor(s) (e.g., remaining supply of solution to be sprayed, temperature and pressure of solution supply, sensors installed for operator and machine safety that sense that protective shielding is in place, machine is in a lowered, operating position, emergency stop buttons are in “off” position, and the like), the second controller sends output signals to control machine operation with respect to a selected plant in the ROI determined to be at a position correlated with action, and produces an implement-actuation command to take a desired action on, or relative to, the plant. Because plant-location boundaries are determined with pixel-level accuracy, the implement-actuation command is also performed at pixel-level resolution.

In another particular embodiment with two controllers, the first or “master” controller is programmed to receive digitized image information, analyze the digitized images with respect to received relative location information of image content, make control decisions based on the analysis of the images, and send electronic control decision outputs to the second controller. The second or “slave” controller receives input, namely relative distance measurement information from the distance measurement device and decision output information from the master controller. Based on this input and on input from other sensor(s) (e.g., sensors installed for operator and machine safety that sense that protective shielding in place, machine is in lowered, operating position, emergency stop buttons are in “off” position, temperature and pressure of solution supply, and the like) the slave controller sends output signals to control machine operation with respect to a selected plant in the ROI determined to be at a position correlated with action, and produce an implement-actuation command to take a desired action on, or relative to, the plant. Based on input information received from master controller, the slave controller also sends a signal the trigger the camera to capture another image.

The system can further include a user interface operably connected to the one or more controllers. In particular embodiments, the user interface is itself considered to be a controller, such as a “master controller.” A user interface allows a user of the system to, inter alia, set parameters useful for particular applications of the system. Exemplary user-adjustable parameters include, but are not limited to, setting length and width of ROIs; setting pixel-to-inch conversion for distance along the trajectory; setting amount of overlap of successive ROIs; setting distance between successive images; setting RGB-to-HSL conversion data; displaying the distribution of plant-representing pixels; setting trailing-edge and leading-edge cutoff levels; setting various plant-spacing parameters such as desired plant spacing in the trajectory, minimum plant spacing, leading-edge buffer distances from plant edges, trailing-edge buffer distances from plant edges, minimum plant length, running average column size in the images, and tolerable noise levels; and performing calibrations of the distance-measuring and image-sensing components of the system. The user interface can also display images produced by the image sensor, and display the output data identifying the location of plants in the ROI.

The user interface can be any computer-input device known in the art. In particular examples, the user interface includes a keyboard, monitor, and mouse. In other examples, the user interface comprises a touch screen. In yet other examples, the user interface comprises a joystick, a bar-code reader, or removable automatically executable storage media device (e.g., a USB drive and the like).

In some examples, the user interface is fixed (mounted) to the support. In other examples, in which the support is pulled or pushed by a vehicle with a driver compartment, the user interface is located in the driver compartment and connected to the controller by standard computer cables, or wirelessly. In still other examples, the user interface can be periodically connected to the one or more controllers by a user, as required, to calibrate and adjust the various parameters of the controller. In yet further examples, the user interface can connect to the controller(s) without a physical connection (i.e. wirelessly) by standard methods known in the art. In such embodiments, the controller(s) further comprises means for producing a wireless signal for connection with the user interface.

### Implements

The system can further include at least one implement, which can constitute a plant-treatment means that is connected to the first controller and/or second controller (if present). The implement receives actuation commands from a controller and in response to the commands executes one or more desired actions. The implement can be any of various devices that “manipulates” or performs an operation on a plant or region associated with the plant. Implement operation is powered by any of various power sources (“drivers”) known in the art, such as a power source that is connected to the controller and that receives the actuation command. For example, the implement may be electrically powered, in which event the controller sends commands to a drive circuit that produces a corresponding drive impulse of sufficient voltage and current to actuate the implement. In another example, the controller commend is received by a pneumatic or hydraulic drive mechanism that correspondingly produces the required flow of fluid to a cylinder or other hydraulic/pneumatic action to actuate the implement. Since the implement-actuation command can be at pixel-level resolution, the resulting action may also be at pixel-level resolution. Exemplary manipulations include, but are not limited to: (a) plant thinning (removal of selected a plant from its detected location in the ROI while leaving other plants in the ROI at their respective locations), (b) weeding (removal of foreign plants), (c) localized (“spot”) cultivating, (d) localized (“spot”) spraying (e.g., pesticide, nutrient, fertilizer, irrigant, herbicide, hormones, acid, base, etc.) or other discharge mode for gases, liquids, solids (e.g., particles, granules, powder), suspensions, or the like, (e) localized plant watering, and (f) localized (“spot”) soil aeration.

In particular embodiments, at least one implement is a pressurized spray system (“sprayer”) that includes a means for providing a pressurized supply of fluid to be sprayed, a means for primary fluid-delivery, an control valve (desirably electrically controlled), a sprayer body, a spray nozzle, and a means for adjusting the angle and profile of fluid discharged from the nozzle. Sprayers are commonly used in agricultural applications, and any spray system known in the art with, but not necessarily limited to, the foregoing components can be used in the systems and methods described herein.

The sprayers and associated hoses and tanks may be used to spray a selected liquid, (e.g., acid, fertilizers, pesticides, herbicides, and the like). Therefore, in particular embodiments, the sprayer is fabricated of a material that is resistant to degradation by the subject liquid or in general various liquid agents used in agriculture.

The sprayer can be used to apply any selected treatment solution suitable for the desired application (including soil, type of target plants, size of target plants, soil characteristics, etc.). In particular examples, the sprayer can be used to apply beneficial treatments to a plant, for example, water, fertilizer, pesticides, fungicides, nematicides, and the like. In other examples, the sprayer can be used to apply a treatment that will selectively kill a treated plant, for example an acid solution (e.g. at 5%, 10%, 15%, 20%, 25%, or greater concentration) or an herbicide.

In particular embodiments, additional sprayers and conventional cultivation tools known in the art can be mounted to or relative to the support and connected so as to be actuatable by the controller. For example, the additional sprayers and/or tools are provided so as to be positioned, during use of the system, outside the plant rows to control weeds in furrows, on bedside walls, and/or between plant rows on the bed. In particular embodiments, multiple nozzles that individually spray different respective chemicals can be mounted to the system so as to be usable in the same row so that a field can be thinned and/or weeded and/or spot treated with pesticides or fertilizers in a single pass over the field.

In embodiments utilizing multiple nozzle and electrically actuated valve assemblies, the multiple assemblies can be positioned so that one or more of them is aligned with the others for treatment of the same plant row. Each of these assemblies can be individually controlled to apply different treatments to respective plants at different distances from a reference location. In one particular example, comprising two nozzle and valve assemblies, a first assembly can be used to spray an agent onto individually thinned plants or onto plants that are weeds, and a second assembly can be used to spray a solution to neutralize and/or minimize the effects of a plant-killing solution previously applied to selected plants. For example, if an acid-based material were used to kill plants, a basic solution can be sprayed on plants to be “saved” (not previously sprayed) to neutralize any acid that may have drifted onto those plants. In another example, water can be sprayed onto “saved” plants by the second assembly to reduce unwanted plant-killing effects of the acid by lowering acid concentrations on the saved plants. Similarly, in examples wherein the first assembly is used to spray a non-acid herbicide to kill plants, water or other diluent can be sprayed by the second assembly to wash away or at least dilute any herbicide that drifted onto the saved plant.

Any of various materials can be mixed with a treatment solution to facilitate the application of the solution to the targeted plant. In particular embodiments, an anti-drift compound is mixed with the treatment solution to reduce drift of the solution during spraying. For example, 3 ounces of polyacrylamide anti-drift material can be added per 100 gallons of treatment solution. Such mixtures are known to reduce drift noticeably in test spraying. In another method, a surfactant is added to improve wetting of the target plant by the spray solution.

In other examples, colored dye marking solutions can be mixed with treatment solutions to provide visible feedback of system performance. Colored dye solutions for marking purposes are commonly available for use with most agricultural chemicals applied in liquid form including pesticides, fertilizers, soil amendments and acids. For example the blue colored SIGNAL™ Spray Colorant (Precision Laboratories Inc., Waukegan, Ill.) can be mixed with a wide variety of herbicides, fertilizers, soil amendments, and acids to mark regions that have been sprayed.

In particular embodiments, the spraying assemblies are attached uncovered to the support. In other embodiments the spraying assemblies can be attached to the support and located in a “hooded sprayer” type assembly that reduces and/or controls over-spraying and/or premature escape of the solution being sprayed. Any of various hooded sprayer assemblies known in the art could be used with the systems described herein.

In other embodiments, at least one implement includes a mechanical blade for killing unwanted plants by digging up the plant or destroying its root. Various components that may be utilized in such embodiments are readily understood by those skilled in the art. In one example, the implement comprises a narrow blade that, when activated, undercuts plant roots whenever the blade is thrust or inserted below the soil surface. A blade implement can include a means for adjusting the blade angle and operating depth.

Blade implements can be driven hydraulically. In a particular example, the implement includes a pneumatic or hydraulic cylinder that is machine controlled through a controlled valve and pressurized fluid supply, or the like, to raise and lower the blade. In other examples, the blade is a linearly actuated knife blade configured to undercut plant roots whenever the blade is inserted or thrust below the soil surface at a target plant. A linearly actuated blade can be supported by a guide shaft, or the like, to provide structural integrity to the blade during use. Means for adjusting blade angle, operating depth, and operation location are readily provided, for example, using a pneumatic or hydraulic cylinder that is machine controlled through a control valve, or the like.

One of skill in the art will appreciate that, in particular embodiments, other or additional implements can be mounted to the support to perform other cultivation actions in response to the command from the controller. For example, weeds can thus be removed between crop rows. Inter-row treatments can be achieved by appropriately adjusting the position of any treatment means. By mounting more than one implement to the support and operating them in a coordinated manner in response to the controller, the system can be used to achieve a desired cultivation goal in only a single pass over the field.

### DESCRIPTION OF PARTICULAR EMBODIMENTS

In the drawings provided herein and described below, it is to be understood that the drawings are exemplary only and are not necessarily shown to scale. Any of various parameters or features described below (for example, size of the support and number, type, and configuration of treatment implements) can be adjusted by one of skill in the art utilizing the present disclosure.

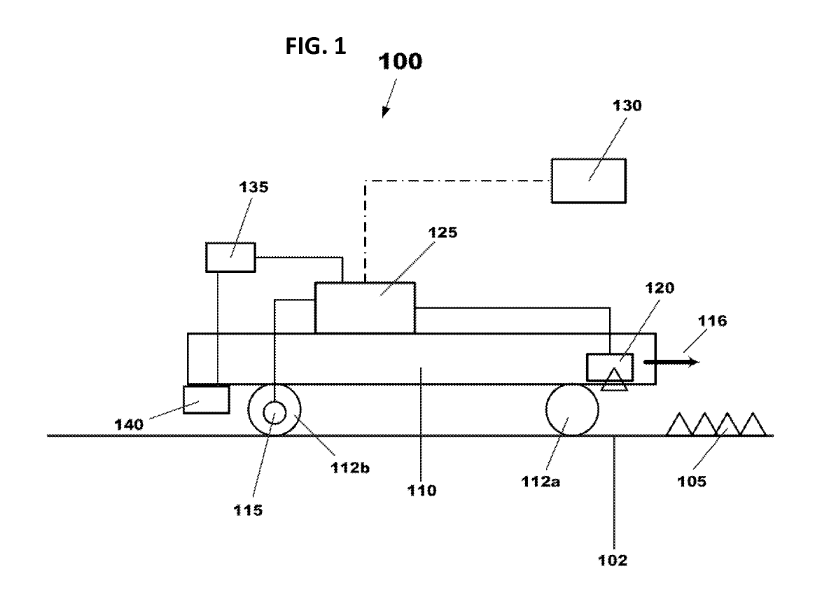

FIG. 1 is a schematic view of an embodiment of a system 100 for automated in situ identification of a plant and for taking selected action on or relative to a plant. The system 100 is shown next to an array of plants 105 (e.g., a crop row). The system includes a movable support 110 to which is connected a distance-measuring device 115 (e.g., optical rotary encoder) for measuring ground distance, traversed by the system 100 as it moves over the ground 102 in a predetermined trajectory 116. Mounted to the support 110 is a machine-vision image-sensor system 120. An exemplary image-sensor system 120 comprises a camera for obtaining, at predetermined distances of the system 100 from a positional reference, digital images of the array of plants 105. Also mounted to the support 110 is a controller 125 that is connected to the image-sensor system 120 and to the distance-measuring device 115. Thus, the controller 125 receives positional information from the distance-measuring device 115. The controller 125 is also connected (at least whenever required) to a user interface 130. The controller 125 is also connected to a power and/or fluid supply 135 that controllably drives operation of an implement 140 (such as a spray nozzle assembly). In the depicted embodiment, the system 100 also includes wheels 112a and 112b by which the support 110 moves over the ground 102. One of the wheels 112b also serves as a ground-following wheel of which rotations are measured by the distance-measuring device 115. In particular examples, the support 110 is held at a fixed height above the ground 102 with an additional set of rotatable wheels (not shown) affixed to the back of the support 110. In other examples, guide cones (not shown) are also affixed to the support 110 to provide support for the main frame and guidance for the implement 140. In still further examples, a linkage system (not shown) connects the support 110 and ground-following wheel 112b such that the implement 140 follows the ground surface.

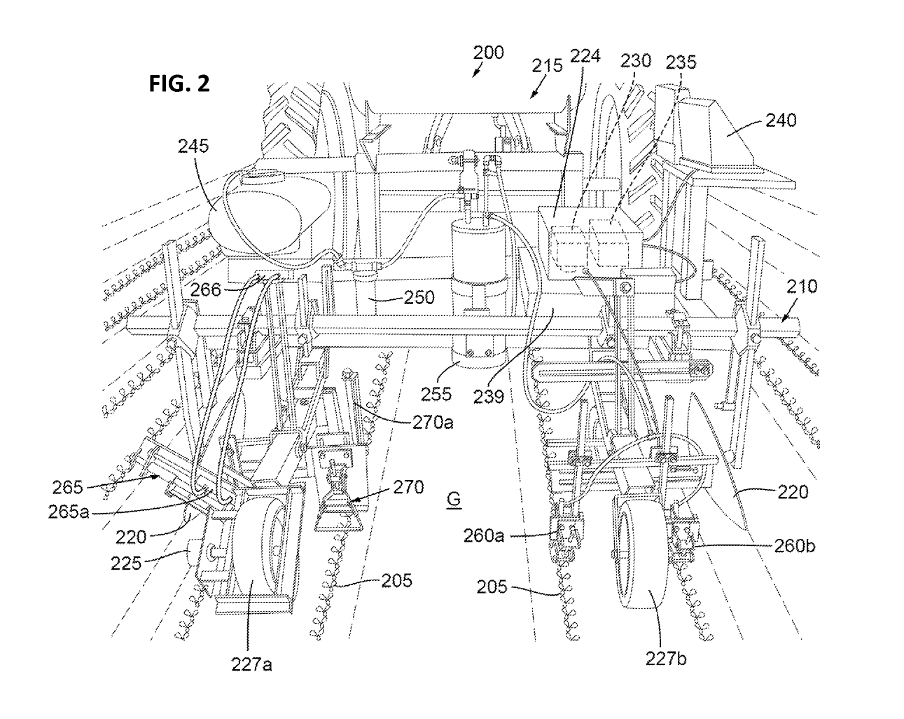

FIG. 2 is a rear view of an embodiment of the disclosed system 200 for automated in situ identification of a plant and for taking action on or relative to a plant. The disclosed system 200 is shown in relation to at least one array of plants 205. The system can include a support 210 that is hitched to and pulled by a tractor 215. Guide cones 220 attached to either or both sides of the support 210 keep the system 200 moving in a desired trajectory along the plant array 205. Mounted to the support 210 are wheels 227a, 227b for lateral and vertical stability. Attached to one wheel 227a is a rotary encoder 225 that “counts” rotations of the wheel 227a as the system 200 moves over the ground G. Also mounted on the support 210, and within an open enclosure 224, are a trigger-activated digital camera (dashed lines) 230 and a computer controller 235. The enclosure 224 is situated atop a light box 239. The light box 239 contains at least one source of light (not detailed) that is directed to the region of the ground G imaged by the camera 230. The light source can be illuminated continuously or intermittently as required to provide light whenever the camera 230 is obtaining an image. The controller 235 is programmed to perform its control functions as described elsewhere herein. The controller 235 is connected to a user interface 240 as described elsewhere herein. Also shown is a tank 245 holding a supply of liquid to be applied controllably to selected plants by the system 200. The tank 245 is connected to deliver the liquid through a filter 250 as urged by a pump 255. Thus, the pump 255 conveys flow of the liquid to spray nozzles 260a and 260b in this embodiment. Also shown are linear 265 and narrow 270 blade treatment assemblies. The blade treatment assemblies 265 and 270 have actuators 265a and 270a, respectively, that are controllably-actuated by respective fluid-control valves (valve 266 for the actuator 265a is shown), or the like, to raise and lower the blades into the soil in the ground G.

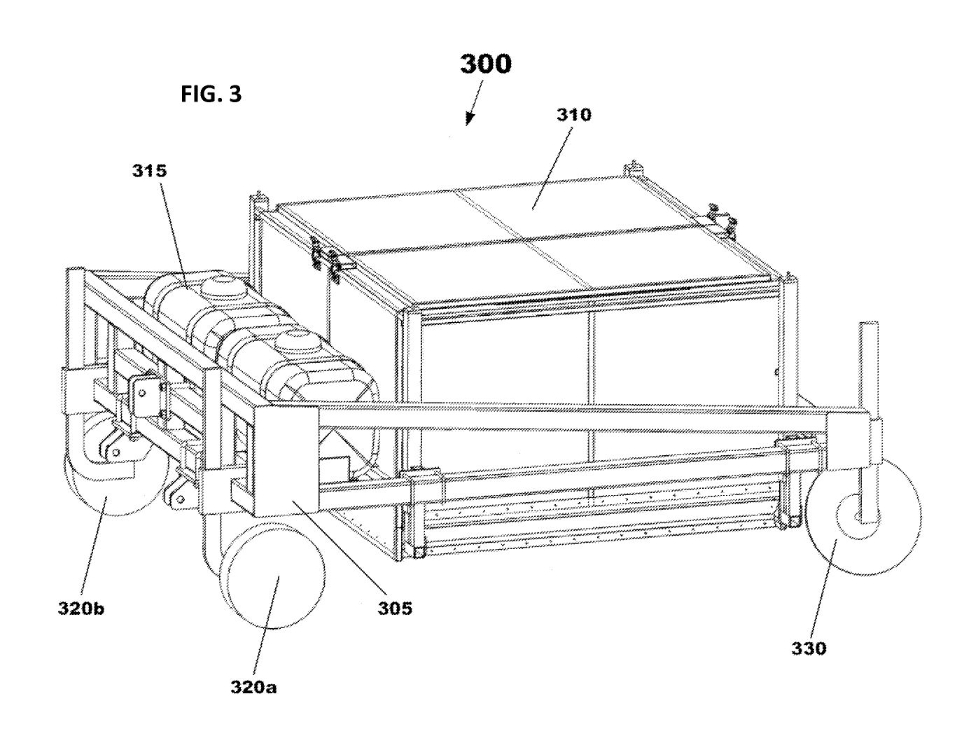

FIG. 3 is a side view of an embodiment of the disclosed system 300 for automated in situ identification of plants and for taking action on or relative to selected plants. Depicted in this embodiment is a support 305 which carries a large enclosure 310 in which the system 300 components are housed. Also shown are tanks 315 for reservoir storage of liquid(s) used in spray treatments. Although not shown in the figure, example components housed in the enclosure 310 are a camera, lights, at least one controller, and implements (treatment means), for example a spray nozzle or blade assembly. The enclosure is provided to shield any sprayed treatment from wind and protect system components from the environment. In the depicted embodiment, the system 300 also includes wheels 320a and 320b by what the support moves over the ground. Also shown is one of at least two guide cones 330 attached to the support 305 to keep the system 300 moving in a desired trajectory along the plant array, as required.

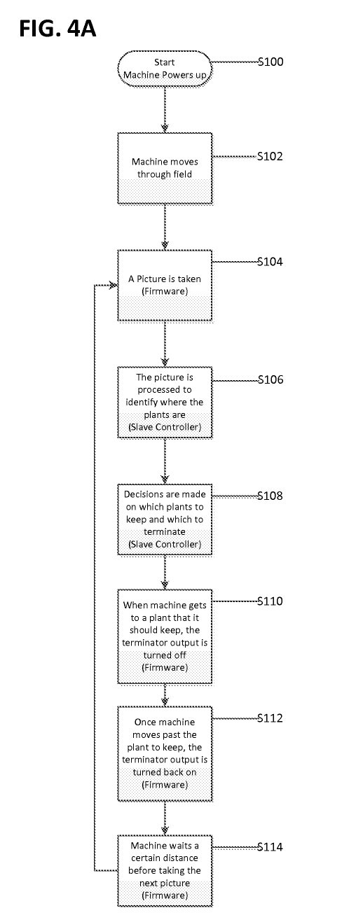

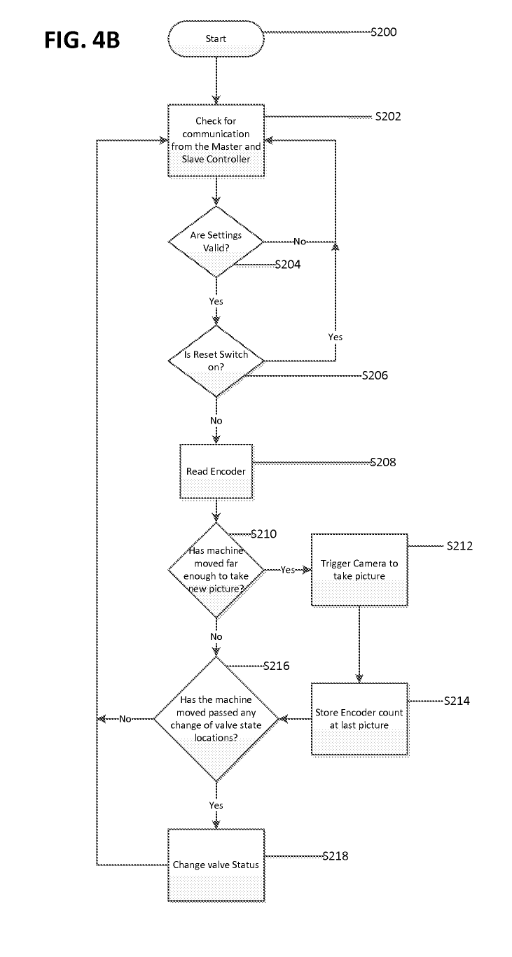

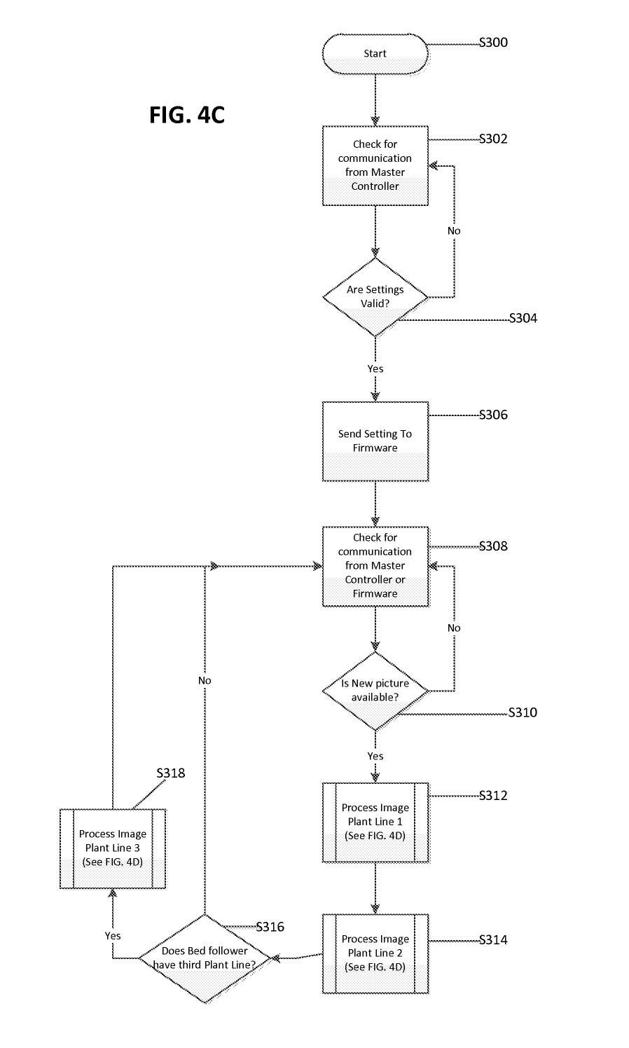

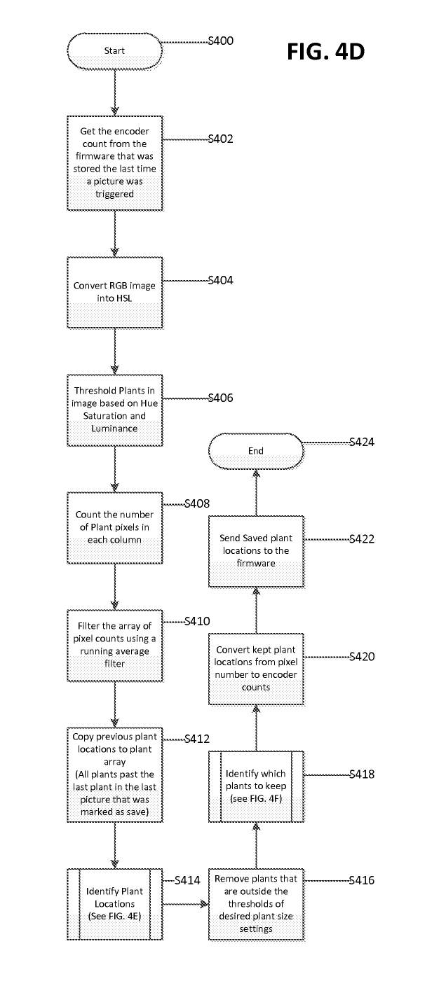

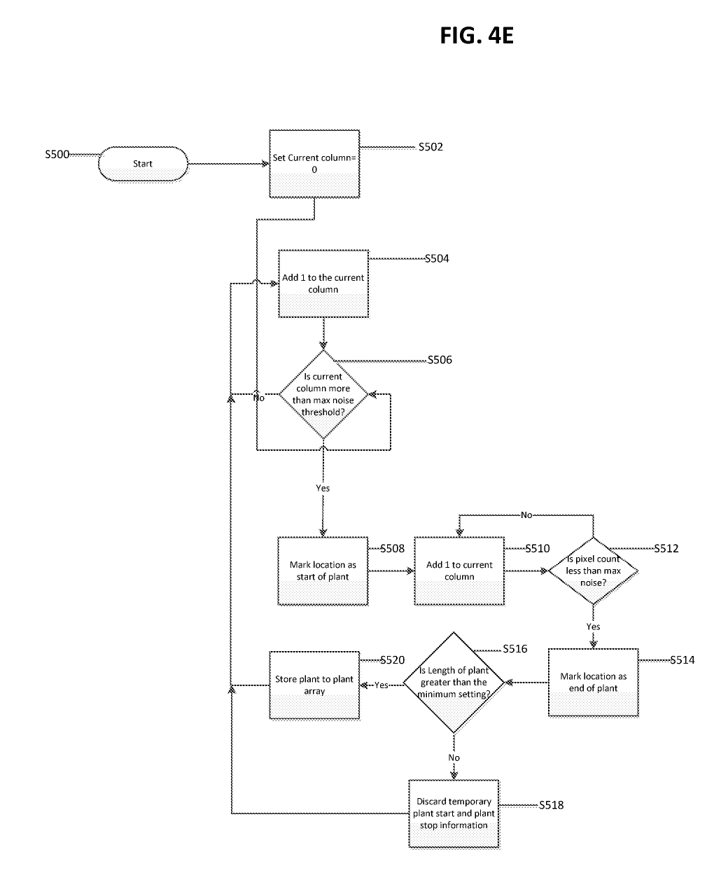

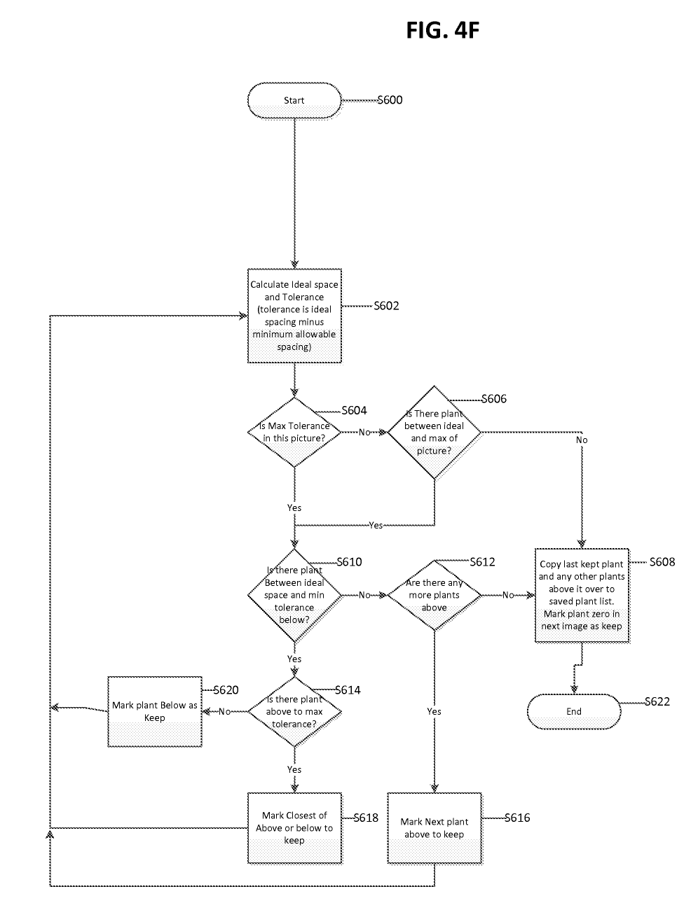

FIG. 4A is an overview of the processes executed by the firmware and software of an embodiment of the described system. System operations start with turning the power on (S100). The system moves through a field (S102), either under its own power, or as a result of being pulled or pushed (e.g. by a tractor). After the system has moved a specified distance, the firmware directs the image sensing device to take a picture (S104). The picture is processed by the slave controller (S106), which identifies plant locations in the picture. Once plants are identified, the slave controller determines which plants to “keep” and which to terminate (S108). When the system is positioned relative to a plant to keep, the firmware turns off any terminator (e.g. sprayer) output (S110). After the system moves past the location of a plant to keep, the firmware turns the terminator output back on (S112). Based on settings programmed in to the firmware, the system waits until it has traveled a specified distance (S114) before signaling the image sensing device to take the next picture (S104).

FIG. 4B is a process flow chart of the operation of the firmware of an embodiment of the disclosed systems. The firmware controls the real-time operations of the disclosed system, including directing the image-sensing device to take a picture and directing terminator output to remove selected plants. As illustrated in FIG. 4B, firmware operation starts with a user turning the system power on (S200). The firmware first checks for communication from the master controller (by way of the slave controller) (S202) and determines whether the settings communicated by the master controller are valid (S204). The firmware next determines if the system reset switch is in the “on” position (S206). The reset switch is automatically moved to the “on” position whenever the machine reaches the end of a crop row and a proximity switch on the machine indicates that the machine has been lifted off the ground as the tractor turns around. If the firmware determines that settings are not valid or if the system reset switch is in the “on” position, the firmware again checks for communication from the master controller (S202). If the reset switch is in the “off” position, the firmware reads the encoder (or any other distance-measuring device) (S208), and the firmware determines if the system has moved the required distance to take a new picture (S210). The required distance between pictures is a setting communicated by the master controller. If the system has moved the designated distance, the firmware triggers the image sensor (e.g., a camera) to take a picture (S212), and the encoder count at the location that the picture is taken is stored (S214). After the encoder count is stored, or if the system has not moved far enough to take a new picture, the firmware determines whether the system has moved past a location designated a “change of valve state location” (S216). A “change of valve state location” is a location correlated with the action of an implement (e.g., either to keep or kill a plant). If the system has passed a “change of valve state location,” an actuation command is sent to the implement to “change valve status” (S218). In the embodiment shown, “change valve status” indicates that the solenoid valve of a sprayer apparatus is triggered to open or close as appropriate. After the “change valve status” command (S218), or if the machine has not moved past a “change of valve state location” (S216), the firmware again checks for communications from the master controller (S202), and restarts the control process.

FIG. 4C is a process flow chart of the operation of the “slave” controller software of an embodiment of the disclosed systems. Slave controller software operation starts with a user turning on the system power (S300). The slave controller first checks for communication from the master controller (S302). The slave controller then determines whether the stored machine operation settings are valid (S304). Machine operation settings include the firmware settings to trigger the camera and the like. If the settings are not valid, the slave controller again checks for communication from the master controller (S302). If the settings are valid, the settings are sent (communicated) to the firmware (S306). The slave controller then checks for communication from the master controller or the firmware (S308) to determine if a new picture is available (S310) for processing. If a new picture is not available, the slave checks again for communication from the master controller or firmware (S308) until a new picture is available. If a new picture is available, the slave processes a first row (“line 1”) of the plant array in the image (S312). The image processing subroutine is described in greater detail in FIG. 4D. In the embodiment illustrated in FIG. 4C, the plant array has at least two rows of plants in the image being processed. Thus, after processing the first row of the plant array in the image (S312), the slave controller processes the second row (“line 2”) of the plant array in the image (S314), by the same subroutine described in FIG. 4D. The slave controller then determines if there is a third row of plants (“line 3”) in the image (S316). If there is not a third plant row, the slave controller checks for communication from the master controller or firmware S308 to determine if a new picture is available for processing (S310). If there is a third plant row, the slave controller then processes that row of the plant array in the image (S318), by the same subroutine described in FIG. 4D. After the third row is processed, the controller checks for communication from the master controller or the firmware to determine whether a new picture is available for processing (S308).

FIG. 4D is a process flow chart showing an embodiment of the “process image” subroutines (S312), (S314), and (S318) of FIG. 4C. In the illustrated embodiment, the “process image” subroutine is executed by the slave computer for each plant “line” (plant row) in the imaging area. The process starts after the slave controller determines that a new picture is available (S400) (see FIG. 4C, S310). The slave controller retrieves the encoder count from the firmware that was stored after the last picture was taken (S402). The image that was taken by the camera (see FIG. 4B, S212) is accessed by the slave controller, and the pixel RGB values are converted to HSL values based on look-up tables (S404). Threshold plants are identified in the image based on the HSL values compared with the settings from the master controller (S406). The controller divides the image into columns one pixel wide, and the number of plant pixels in each column of the plant row being processed are counted and stored in a one-dimensional array (S408). To remove high-frequency spikes from the distribution, the slave controller filters the array of pixel counts using a running average filter (S410) (also a setting communicated by the master controller). The slave controller then copies the previous plant locations to the plant array (S412); this includes all plants past the last plant in the last picture that was marked as “save.” The slave controller next identifies plant locations in the row being processed (S414), by the subroutine illustrated in FIG. 4E. Once plants are identified in the row, the slave controller removes from the list of plants to save those plants that are outside the threshold of the desired plant size settings (S416). The slave controller next identifies which plants to keep (S418), by the subroutine illustrated in FIG. 4F. Once the plants to keep are identified, the locations of the kept plants are converted from pixel number to encoder counts (S420). The slave controller then sends the locations of plants to be saved to the firmware (S422). The subroutine ends (S424) with the slave controller checking for the availability of a new picture (or plant row) to be processed.

FIG. 4E is a process flow chart showing an embodiment of the “identify plant locations” subroutine S414 of FIG. 4D. The subroutine starts (S500) after the slave controller copies the previously identified plant locations to the plant array (see FIG. 4D, S412). The slave controller sets the first column of the pixel array being analyzed as “0” (S502). The slave controller next determines whether the number of pixels in the column is more than the maximum noise threshold, as determined by settings from the master controller (S506). If the number of pixels is not more than the maximum noise threshold, the slave controller adds 1 to the identity of the column to be analyzed (S504) (thereby analyzing the next adjacent column) and again determines whether the number of pixels in the column is more than the maximum noise threshold (S506). If the slave controller determines that the number of pixels is more than the maximum noise threshold, the slave controller marks the column location as the start of a plant (S508). The slave controller then adds 1 to the identity of the column to be analyzed (S510) (thereby analyzing the next adjacent column), and determines whether the number of pixels in the column is less than the maximum noise threshold (S512). If the number of pixels in the column is not less than the maximum noise threshold, the slave controller then adds 1 the identity of the column to be analyzed (S510) (thereby analyzing the next adjacent column), and determines whether the number of pixels in the column is less than the maximum noise threshold (S512). This process is repeated until the slave controller determines that the number of pixels in the column is less than the maximum noise threshold, and the slave controller marks the column as the end of the plant (S514). The slave controller then determines whether the plant length is greater than the minimum plant-length setting (as communicated by the master controller) (S516). If the plant length is not greater than the minimum plant length, the plant start and stop information is discarded (S518). If the plant length is greater than the minimum plant length, then the plant is stored to the plant array (S520). Whether or not the plant is greater than the minimum plant length, the slave controller then adds 1 to the identity of the column to be analyzed (S504) (thereby analyzing the next adjacent column) and repeats the subroutine until the last column of the plant row.

FIG. 4F is a process flow chart showing an embodiment of the “identify which plants to keep” subroutine S418 of FIG. 4D. The subroutine starts (S600) after the slave controller removes those plants that are outside the threshold of the desired plant settings from the list of identified plants (see FIG. 4D, S416). The slave controller first calculates the desired distance between plants (“ideal distance”) and the “tolerance,” which is the ideal distance between plants minus the minimum distance allowable between plants (S602). The controller then determines whether the maximum tolerance (ideal distance plus tolerance) is in the picture (S604). If the maximum tolerance is not in the picture, the controller determines whether there is a plant between the ideal distance and the end of the plant row under analysis (S606). If not, then the controller copies the last kept plant (marked “saved”) and any other plant located above it (after it in the plant row), onto the saved plant list. The first plant in the next image (plant 0) is marked as saved (“keep”) (S608) and the subroutine ends (S622). If the controller determines that a maximum tolerance (ideal distance plus tolerance) is in the picture (S604) or that there is a plant between the ideal distance and the end of the plant row (S606), then the controller determines whether there is a plant between the ideal distance and the minimum tolerance distance (ideal distance minus tolerance) (S610). If not, then the controller determines whether there are any plants above the ideal distance (S612). If not, then the controller copies the last plant kept onto the saved plant list (S608) and the subroutine ends (S622). If there are any plants above the ideal distance (S612), the controller marks the next plant above as “keep” (S616) and returns to the start of the analysis (S602). If there is a plant between the ideal distance and the minimum tolerance distance (S610), the controller determines whether there is an additional plant after it in the row up to the maximum tolerance distance (S614). If not, the controller marks the plant identified below that is situated between the ideal distance and the minimum tolerance distance to be saved (S620), and the controller recalculates the ideal distance and tolerance from the saved plant to restart the analysis (S602). If there is a plant above the ideal distance, up to the maximum tolerance distance (S614), then the controller determines which plant (the one above or below the ideal distance) is closer to the ideal distance, and the controller marks that plant to be saved (S618). The controller then recalculates the ideal distance and tolerance from the saved plant and the analysis begins again (S602).

### Methods of Automated Identification of and Performance of an Action on a Plant

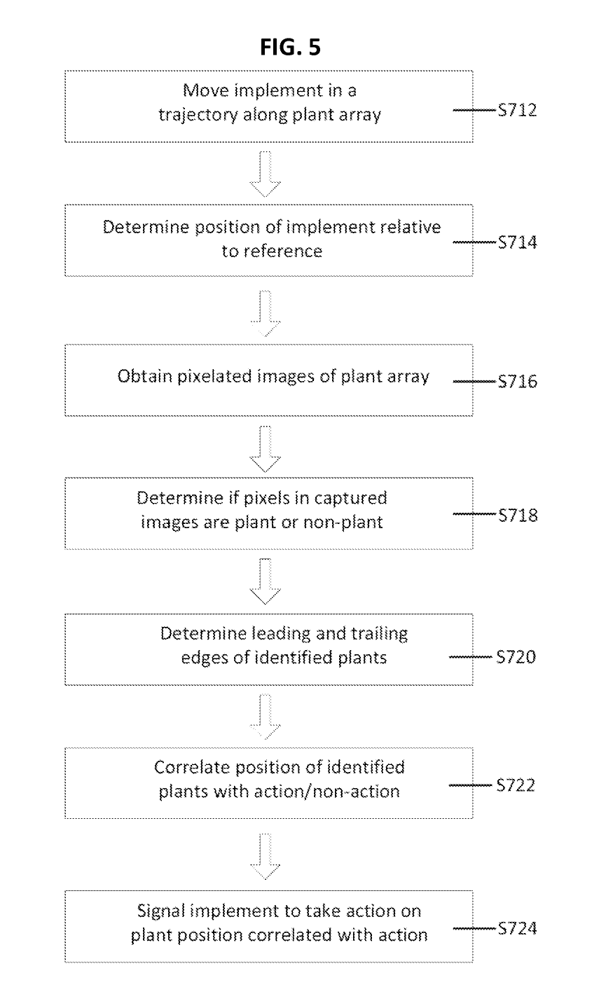

Disclosed herein are methods of in-field, real time identification of plants, and performance of one or more selected actions on the identified plant. The described methods are carried out using a machine-vision assisted plant-identification system as described above. An overview of an exemplary method of manipulating plants in situ is set forth in FIG. 5. In the first step of the presented method, the system, which includes at least one implement, moves along a trajectory relative to an array of plants (S712). The machine-vision system next determines the position of the system (and thus, of any implement associated therewith) in real time relative to a positional reference along the trajectory (S714). While moving the implement, the system obtains, in real time, a series of pixelated images of respective portions of the array located in respective regions of interest (ROI) situated at discrete respective distances from the positional reference (S716). In each pixelated image, the system determines whether respective image light received at each of the pixels is indicative of plant versus non-plant (S718). In each pixelated image, the system determines respective leading and trailing edges of plant-indicating pixels (S720) and correlates these positions with a desired action or non-action to be taken with respect to selected plants in the respective ROI (S722). Lastly, the system signals the implement to take action with respect to a plant in the ROI determined to be at a position correlated with the action (S724).

The exemplary method described above has been tested in fields containing several varieties of head lettuce and one type of Romaine lettuce. The method proved to be effective at identifying plants in each of the types of lettuce crops tested. The method differentiated between lettuce and weed plants. For example, one of skill in the relevant art will understand that the systems described herein can identify and/or distinguish virtually any crop plant. The system also can differentiate, in most instances, between crop plants and weed species.

In the various embodiments, the machine-vision systems are used in the described methods of identifying and taking action on plants in any of various agricultural situations such as a crop field typically used for growing crop plants planted in earth. Alternative agricultural situations include, but are not limited to, plant nurseries, arrays of plants grown in individual containers, plants germinated in germination arrays, etc., and hydroponic arrays. In particular examples, the plants in the given agricultural setting are arrayed linearly. In other examples, the plants to be detected and acted on can be arrayed in a non-linear manner, so long as positional (distance) measurements are possible. However situated, the array of plants should be in the normal movement direction of the vehicle that moves the system relative to the array. In examples wherein crops are planted in mostly linear rows, only portions of the image need to be analyzed. These image portions, termed regions of interest (ROI) can be defined by the user through a program that runs on the user interface.

In an exemplary embodiment, the controller is programmed to identify plants on a pixel level as follows. The imaging system (machine vision) camera captures digital images in standard red (R), green (G), blue (B) format so that each pixel in the captured image has associated R, G, and B (RGB) values (S212). The controller is programmed to convert these values to corresponding hue (H), saturation (S), and luminance (L) values (S404). These values are individually compared with user-adjustable, preset threshold maximum and minimum H, S, and L (HSL) values to determine whether or not a pixel represents part of a subject plant (S406). For each pixel, the controller determines whether light received at the pixel is above or below the preset threshold. If the light is above the threshold, the pixel is considered as displaying a part of a plant. If light is below the threshold, the pixel is considered not to be displaying a respective part of a plant. The user interface can display pixels as plant or non-plant by any way that distinguishes the two pixel types, such as different colors or the like. In a particular example; plant pixels are displayed as white and non-plant pixels are displayed as black.

One of skill in the relevant art will appreciate that RGB to HSL conversion is not the only way in which identifications can be made of whether a given pixel is indicative of plant versus non-plant. RGB conversion typically starts with a color image, wherein conversion to HSL is a way of converting the color image (in which each pixel could have any of a large number of states) to a binary image (in which each pixel has either one state or another state). In particular examples, instead of the controller converting RGB values to HSL values, the camera is capable of converting RGB values into HSL values. In another example, an image can be obtained at a single wavelength that may eliminate the need to do a color-to-binary conversion. For example, the wavelength could be a key wavelength associated with photosynthesis or a wavelength distinctive to the subject plants.

The pixels in a captured image are identified as plant or non-plant by analysis of the ROI. In particular examples, the ROI is of predetermined dimensions. In other examples, the ROI parameters (including dimensions) are changed by the user using the user interface.

Once the pixels in an ROI are determined to be plant or non-plant, the controller determines the distribution of pixels in a ROI by creating, for each image, a frequency-distribution plot of “plant” pixels in each column of pixels in the image, across all rows of pixels in the image, versus distance (S408). Distance can be in terms of column width (one pixel), yielding a distribution at pixel-level resolution. Alternatively, distance can be in the same units (e.g., inches) utilized by the distance-measuring device of the systems described herein. By analysis of the distribution of plant pixels, trailing and leading edges of plant regions are determined without having to perform higher-level processing of the pixelated images (S414).

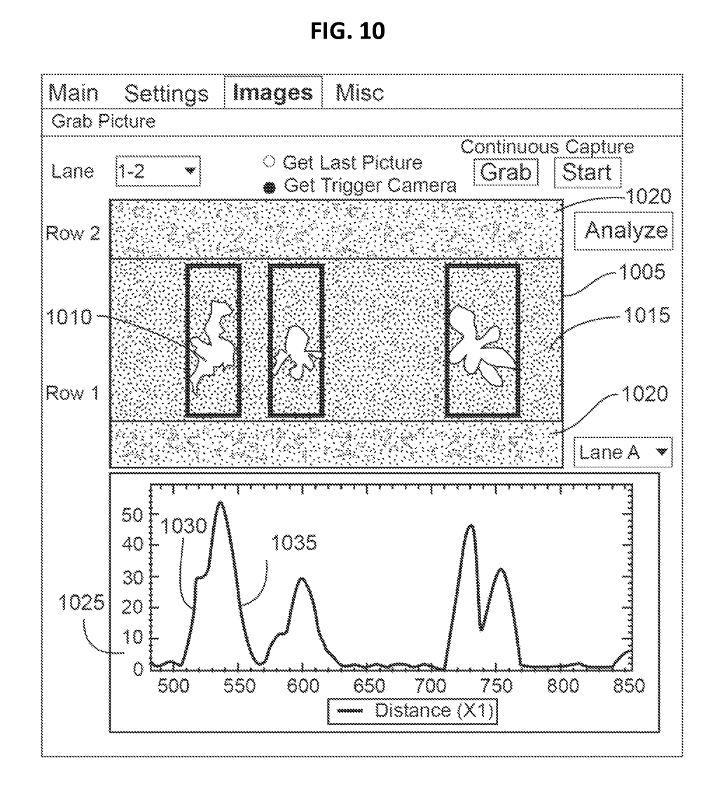

The systems described herein can identify a plant in a captured image by analyzing pixels present in a ROI of an image. The controller is programmed to analyze the pixels in the ROI as follows. An exemplary analysis is depicted in FIG. 10, which depicts an exemplary screen display of the user interface. Each pixel within the ROI 1005 is converted from color to white and black based on the HSL intensity analysis described above. In FIG. 10 (top), sections of the ROI determined to be “plant” pixels are white 1010, non-plant sections of the ROI are speckled 1015. The controller then divides the ROI 1005 into an array of horizontal rows and vertical columns, wherein each cell in the array is the size of a single pixel in the ROI 1005. The controller tabulates the number of “plant” pixels in each column and stores this information in an array in memory. To reduce problems caused by “spikes” of pixel counts in individual columns, a running average system can be employed to assign pixel counts for each column. The number of columns of pixels used for this averaging technique is a user-adjustable parameter and is termed the “array filter size”. Because pixel width and length represent “real-world” physical distances, frequency distributions of plant parts (white pixels) can be plotted versus distance. The lower portion of FIG. 10 shows an exemplary pixel distribution plot 1025, wherein the number of pixels in each column across all rows within the ROI 1005 are plotted versus distance in terms of column width. In FIG. 10, one pixel is about 0.024 inches wide, in other examples, one pixel can be 0.125 inches wide.

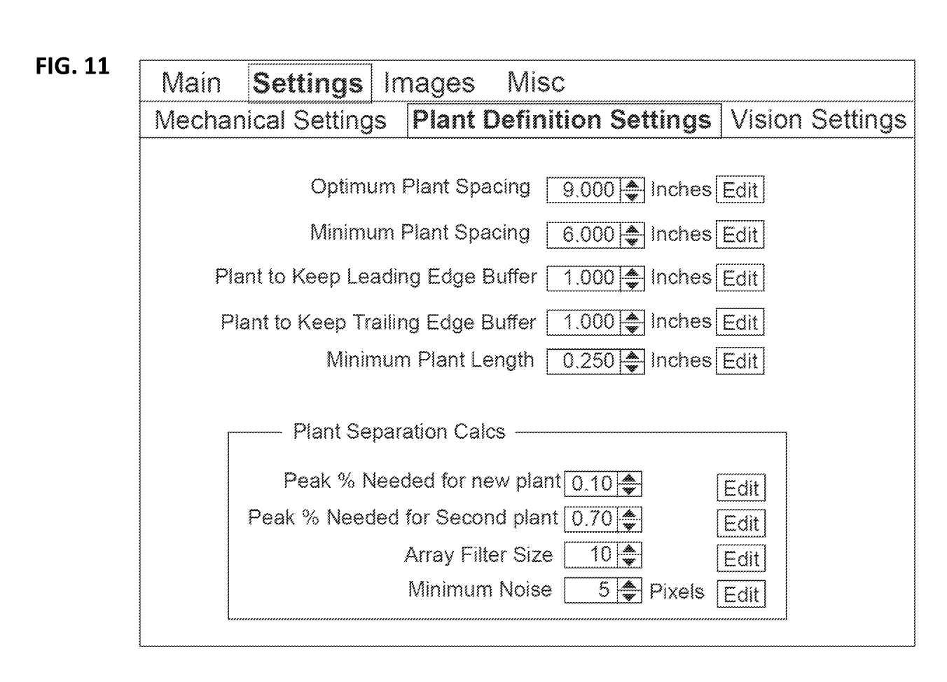

A plant in a given ROI is identified when more than a predefined number of adjacent columns (“minimum plant length”) each contain at least one white pixel more than a predefined number of white pixels (“noise” value). In particular examples, “minimum plant length” and “noise” are pre-set or standard values. In other examples, one or both of these are user-determined and set through the user interface (see FIG. 11). In some examples, the minimum plant length is input as inches or millimeters; in other examples, minimum plant length is entered in inches or millimeters and converted to corresponding number of columns; in still other examples, minimum plant length is entered as number of columns in the plant pixel distribution.

Once a plant is identified, the left-most column (see for example 1030 in FIG. 10) in the array of adjacent columns is taken to be the trailing-edge location while the right-most column (1035, for example) is taken to be the leading-edge location. In one example (depicted in FIG. 11), minimum plant length can be set to 0.25 inches, a noise level set to 5 pixels and an array filter size set to 10 pixels. In other examples, minimum plant length can be 0.75 inches, noise level can be 10 pixels, and the array filter size can be 20 pixels.

In particular examples, similar programming logic can be used to determine the location of non-horizontal plant boundaries to provide additional geometrical plant characteristics. For instance, “top” and “bottom” plant edges in the vertical direction used either alone or in combination with “leading” and/or “trailing” plant edges in the horizontal direction. In some examples, non-horizontal values can be used in conjunction with plant length to obtain a more accurate estimation of plant diameter than just plant length. Such analysis can be accomplished for example, by computing the length of a line connecting the opposite diagonal corners of a rectangle drawn around the “plant-defined” section of the ROI. In other examples, computation of plant center locations in both horizontal and vertical directions can be used to automatically center the ROI over each seed row. In other embodiments the controller is additionally programmed to compute plant cross-sectional area by summing all identified plant part pixels.

In a particular embodiment, the determination of several plant features allows for selection of plants based on a combination of one or more geometric attributes including length, width, diameter, cross-sectional area and/or ratios or combinations of any other foregoing parameter. In particular examples, such features can also be used to develop algorithms to differentiate crop plants from weeds or select crop plants with preferred characteristics. In particular examples, the subject crop is a lettuce or similar broad-leaf plant with oval shaped leaves, which can be geometrically differentiated from grassy type weeds with long, narrow leaves. In other examples, the crop has long, narrow leaves, and can be differentiated from broad-leaf type weeds.

In still other embodiments, plant-center locations can be used to automatically control lateral alignment of any machine component so that it is centered over the crop row or situated as to guide a vehicle automatically along the crop row. In particular examples, lateral alignment is achieved in conjunction with a global positioning system (GPS), such as in the iGuide™ alignment system (John Deere, Moline, Ill.).

Once a plant is identified and its trailing and leading edge positions are located, the controller is programmed to compute the center (horizontal midpoint) of the plant and establish independent “buffer” zones in front of and behind these edges (S602). In particular examples, the buffer zones are based on user inputted distance values. In other examples, the buffer zones are pre-set standard values. Such buffer zones are termed the “trailing edge buffer distance” (TEbd) and “leading edge buffer distance” (LEbd). Once computed, the buffer zones are stored in the memory of the controller. The program then searches the analyzed ROI to identify the next plant to be “saved” based on TEbd, LEbd, desired plant spacing (D_{desired}) and minimum plant spacing distances (Dₘᵢₙ) (S418). Like the buffer distances, D_{desired} and Dₘᵢₙ can either be user-inputted or pre-set “standard” values. The next plant to be saved is identified as the one whose center is located at a distance from the “already saved” plant that is greater than the Dₘᵢₙ, and at a distance that is closest to D_{desired}. Once the next plant to be saved has been selected, TEbd and LEbd for this plant are calculated, and are stored in memory. The process is repeated until the entire ROI is analyzed. Then the next ROI is similarly evaluated.

The buffer distances need not be limited to the horizontal direction. In particular embodiments, the controller can be programmed to determine buffer distances in vertical or radial (circular or elliptical shaped) directions from the plant row. In another example, a vertical buffer distance can be programmed to control the treated distance perpendicular to the plant row. In such examples, precision weeding treatments can be provided close to the plant row, but far enough way in the perpendicular direction as to not injure the crop plants.

Establishment of TEbd and LEbd between adjacent plants allows for selective treatment outputs to be controlled based on machine position (S216 and S218). For example in weeding and thinning embodiments, a plant-terminating implement can be activated at the right edge of the leading edge buffer (LEb) of a saved plant and stopped when the plant terminating implement has reached the left edge of the trailing edge buffer (TEb) of the next plant to be “saved.” The area in which a selective treatment is given is referred to herein as the “distance treated” (D_{treated}).

It is understood in the art that commonly used sowing machines are prone to picking up and dropping two or more seeds at the same time. Groupings of such plants as germinated in the field are commonly referred to as “doubles.” In particular examples of the systems described herein, the controller is programmed to direct selective thinning of adjacent rows of crop plants such that the remaining plants are equidistant from each other. Although such thinning is commonly done by hand and termed a “diamond pattern,” the systems disclosed herein can be programmed for “diamond pattern” thinning based on estimations of plant centers. The advantage of the diamond pattern thinning technique is that because individual plants require a minimum sized area for optimum yield and plant spacing is preferably equidistant, crop plant density and therefore crop yield are maximized.

The methods described herein employ one or more implements for thinning, weeding, or other manual operations directed against target plants. For example, a plant-terminating implement, such as a blade or a sprayer, can be used to thin and/or weed between plants. During the same pass through the crop stand, a second implement, such as a sprayer, can also be used to apply a selected liquid product such as a pesticide, a growth regulator, or even water. One of skill will appreciate that additional implements can be added to the described system as desired. Performance of multiple operations on a plant stand in a single pass with the described methods has the added benefit of reducing soil compaction due to multiple passes over the field.

### Calibration Methods

The methods described herein require the physical distance of each pixel in the captured image to be calibrated accurately to physical, real-world dimensions. Therefore, provided herein are methods of calibrating the described machine-vision systems. Exemplary methods of such calibration involve detection and distance measurement of alternating colored stripes of known width and length. Any such pattern that is detectable by the described systems can be used to calibrate pixel size with the real-world distance.

In a particular example, a “calibration board” of alternating black and white strips of known width (e.g. 0.5, 1, 2, 3, 4 or more inches wide) is placed on the soil surface. The height of the strips above the soil is adjusted such that they at the same height as the maximum cross sectional area of the crop plants. The described imaging system is used to capture an image of the calibration board, and the controller determines the location of each white and black pixel in the image. Because the distance between strips is known and the number of pixels in horizontal and vertical directions is a function of the particular camera used, and is of known value, the number of pixels per linear inch in the horizontal and vertical directions can be determined. In other examples, instead of the “calibration board,” strips of paint can be sprayed directly on the soil at known distance intervals.

Calibration of image pixels with real-world distances allows for accurate calculation of real-world plant features including plant-leaf edge locations and distances between plant midpoints. Such measurements are necessary for selective action on a given plant or area around the plant, including but not limited to selective thinning, selective weeding, selective spraying, establishing non-treated buffer distances, and combinations thereof.

The methods described herein allow the system to be calibrated to real-world objects that are positioned at the same distance from the camera as the calibration surface during the calibration procedure. In particular embodiments, calibration is maintained by mounting the camera on a floating, ground-following device positioned on the top of the crop bed top or close to the plants of interest. Thus, the camera remains positioned at a relatively constant distance from the plants of interest and thus helps keep the system in calibration. In other examples, the calibration surface, such as a calibration board, is mounted on a floating, ground-following device positioned on the crop bed top or close to the plants of interest. The controller is then programmed to analyze the portion of captured images periodically where the calibration board is located and automatically update calibration constants as the machine travels through the field. A running average of determined calibration constants can be programmed to optimize calibration accuracy and minimize potential errors in overall system performance due to individual calibration constant outlier data. In another example, the calibration board is replaced with colored strips of known length that are sprayed on the soil surface close to the plants of interest. The color of the dye used is one that can be easily distinguished by the vision system from the soil surface. In particular embodiments, strip length can be correlated with a particular distance measured by the distance-measuring device, such as encoder pulses. In such examples, the encoder is configured to signal the solenoid valve to spray the dye after a specified number of encoder pulses. Images of the sprayed strips can then be analyzed using the described calibration procedures to estimate image pixel size in real-world dimensions (pixels/inch).

The following examples are provided to illustrate certain particular features and/or embodiments. These examples should not be construed to limit the invention to the particular features or embodiments described.

### EXAMPLES

### Example 1

### Machine-Vision System for Automated Plant Identification and Selective Thinning, Weeding and Spraying

This example describes an exemplary machine vision system for automated, in-field plant identification and taking selective action on a plant or plant area.

A system for automated in field identification of a plant was constructed as illustrated in FIG. 2. The system is composed of a steel frame 210 hitched to a tractor 215, which pulls the system through a field of plants. The other components of the system are mounted to the frame. Distance and position measurement is accomplished by an optical shaft encoder 225 directly attached to the axle of a rotatable wheel 227a of the frame 210. As the system moves over ground G, the wheel 227a rotates correspondingly as the encoder 225 counts rotations of the wheel 227a. In the model used in this example, the encoder resolution is 1000 pulses per revolution and the wheel diameter is 11 inches. Therefore, the position of system components can be determined with an accuracy of 0.035 inches under ideal conditions (in which the wheel 227a does not slip relative to the ground). Such accuracy is sufficient for most agricultural applications.

Mounted to the frame 210 and housed within a box 224 is a trigger-activated, high-speed, high-resolution digital camera 230 (Model DFK41BU02H, The Imaging Source, Charlotte, N.C.). The camera can capture an image with a resolution of 1360 pixels in length×1024 pixels in width, using an RGB CCD sensor within 4.8 micro seconds (0.0000048 seconds) of receiving an electrical “trigger” signal between 3.3 and 12 V. In this example, the camera 230 is “triggered” (i.e., sent a 12 V electrical signal) whenever the machine moves a preset “distance between pictures,” typically about 21 inches. The distance measurement is performed by the optical encoder 225. Thus, when the controller 235 (housed in the example within the same box 224 as the camera 230) receives a given number of electrical pulses from the encoder 225, an electrical signal is sent by the controller 235 to activate the camera 230 to obtain an image.

The camera 230 was positioned at a height such that its field of view matched the inner dimensions of the open-bottomed box 239 positioned below the camera. The depicted box not only supported the camera, but also provided controlled lighting conditions for obtaining good images. In this example, fluorescent tube lights were used for illumination. Specifically, six lamps, each 18 inches in length, were mounted to the inner top of the box 239 and arranged to distribute light uniformly. Total bulb energy output was 76 Watts, as four of the lamps provided 15 Watts power while two of the lamps provided eight Watts. Ambient light was minimized by attaching an approximately 1 millimeter-thick thin rubber skirt to the bottom of the box.

Two controllers (shown as a single feature 230) (“computers”) were housed in the same box 239 as the camera. The first controller (Fit PC2, Compulab, Haifa, Israel) receives digitized image information, analyzed the digitized images, made control decisions based on the analysis of the image, and sent electronic control decision output to the second computer. The second controller (custom-built) received this input; and based on this input as well as input regarding position of the treatment means in relation to the selected plants, the second controller sent output signals to control action taken by the selected implements at a specified position. The controllers were programmed and the collected data were visualized by the user interface 240, also mounted to the frame.

In the system illustrated in FIG. 2, the implements comprised two solenoid spray nozzles 260a and 260b and two knife-blade cultivators 265 and 270. However, in the following examples, only the spray implement is utilized for selectively thinning lettuce seedlings.

### Example 2

### Calibration of Distance Measurement

This example describes several procedures for calibrating distance measurement by the automated system described in Example 1.

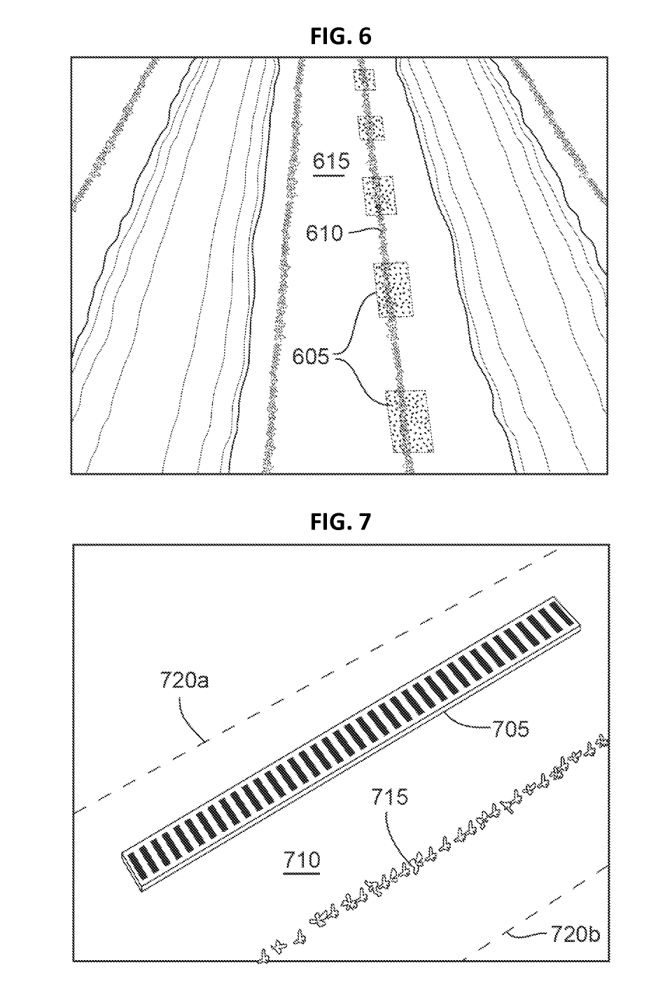

One limitation on the accuracy of a method based on a ground-driven wheel encoder for determining machine position is wheel slip may occur, depending upon soil surface conditions, which are not constant. To minimize this possible error, the described system is programmed to allow the user to adjust the distance-measurement calibration in the field. This is achieved through standard techniques of counting the number of encoder pulses output over a given travel distance and inputting the ratio of these two numbers (number of pulses/inch). Alternatively, a spray nozzle was attached to the machine support and used to alternatively spray and then not spray a colored dye solution for a given number of encoder pulses. Spray is controlled through a solenoid-activated valve. Based on the wheel diameter of the ground driven wheel, the system was programmed to spray “strips” approximately 12 inches long and 12 inches apart (605, as depicted in FIG. 6). The strips 605 were sprayed in situ along a plant array 610 situated on a crop bed 615. The distance between five strips (approximately 10 feet) was measured and input into the controller via the user interface. The program then calculated the actual inches/pulse and stores the value in memory. Accuracy was verified by repeating the procedure.

### Example 3

### Calibration of Real-World Distances with Pixels in Captured Images

This example describes several procedures for calibrating real-world distances with pixels in the images captured by the automated system described in Example 1.

To enable the described system to locate and take action on a plant accurately, the physical distance of each pixel in the images captured by the camera was calibrated to physical, real-world dimensions.

As depicted in FIG. 7, calibration of real-world distances with pixel size was accomplished using a “calibration board” of alternating black and white strips 705, each one-inch wide. The calibration board 705 was placed on the soil surface 710 on top of a crop row. FIG. 7 depicts the crop row boundaries 720a and 720b. The height of the strips above the soil was adjusted to be positioned at the same height as the maximum cross sectional area of the crop plants 715. The machine vision system described in Example 1 was used to capture an image of the calibration board 705 and determine the location of each white and black pixel in the image. Because the distance between strips is known (1 inch) and the number of pixels in horizontal and vertical directions are determined by the camera used, and are of known value, the number of pixels per linear inch in the horizontal and vertical directions could be determined.

Many camera lenses distort true distances at the peripheral edges of an image produced by the lense. Therefore, the analyzed pixel data from the calibration board and the distance of said pixels from the center of the image were used to perform a best fit cubic regression and obtain a cubic equation to predict the physical distance of one image pixel in horizontal and vertical directions from the center of the image.

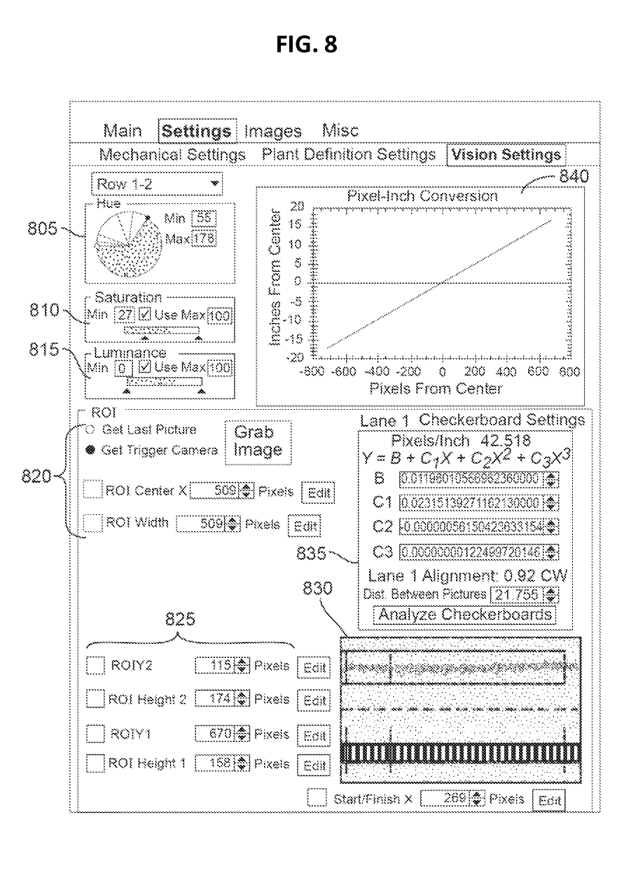

The above calibration procedure is illustrated in the user-interface screen depicted in FIG. 8. The screen shot illustrates user-adjustable fields for minimum and maximum hue 805, saturation 810, and luminance 815 pixel values. Also shown are input fields to adjust the size of image obtained 820, and adjust the size of the ROI analyzed 825. The lower right corner of the figure shows the analyzed captured image of the crop row and calibration board 830. An analysis window 835 is shown above the captured image 830. In the analysis window 835, the best fit cubic regression constants were fit to the pixel data, yielding a calibration value of image pixel distance to real-world physical distance of 42.518 pixels/inch. The graph 840 in the upper right corner reveals how well the cubic regression predication equation determined by the program performed across the length of the ROI selected. The more linear the line in the graph, the less camera distortion that is present. This visual representation is helpful for calibrating the machine in that problems due to human errors are easily recognized. For example, if the calibration board is not mostly horizontal or if portions of it are outside the ROI, the cubic regression plot will appear obviously nonlinear.

With pixels per real-world inch in the image being accurately calibrated, real-world distances between pixels determined to represent plant geometrical features can be calculated from locations of plant leaf-edges. Also, distances between plant midpoints can be calculated and used for a variety of purposes including, but not limited to, selective thinning, selective weeding, selective spraying, establishing non-treated buffer distances, and combinations thereof.

### Example 4

### Encoder-Based Calibration

This example describes an alternative method of calibrating the pixel size in captured images with real-world distance, using pulses of an encoder as a unit of measurement.

Through a procedure similar to the one previously described, image pixel dimensions are determined in terms of pixels per number of encoder pulses. Use of this value allows for very accurate correlation between machine movement and pixel image location. The error is limited because the determined value of pixels per number of encoder pulses is dimensionless. Errors associated with physical distance measurement (pulses/inch) and pixel dimensions (pixels/inch) calibrations are eliminated. Additionally, errors in encoder-distance calibration due to wheel slip would induce treatment means errors that are small and of negligible consequence. This may be illustrated by the example in which the system is programmed to thin lettuce seedlings nominally spaced 2 inches apart to a final spacing of 11 inches. Nominal plant length is ¾ of an inch and trailing- and leading-edge buffer distances are set to 1.5 inches. All dimensions are converted by the program to “encoder pulse” units. With an encoder calibration value of 28 pulses per inch, one inch is converted to 0.0357 pulses. If wheel slip causes the actual encoder calibration to be lowered by 5% and there are no other system errors, the trailing- and leading-edge buffer distances are 1.43 inches, and selected plants would be nominally thinned to 10.5 inches. As compared to preset values, these errors are of negligible practical consequence.

### Example 5

### Pixel-Level Plant Identification

This example describes use of the described machine vision system to identify plants with pixel-level accuracy.

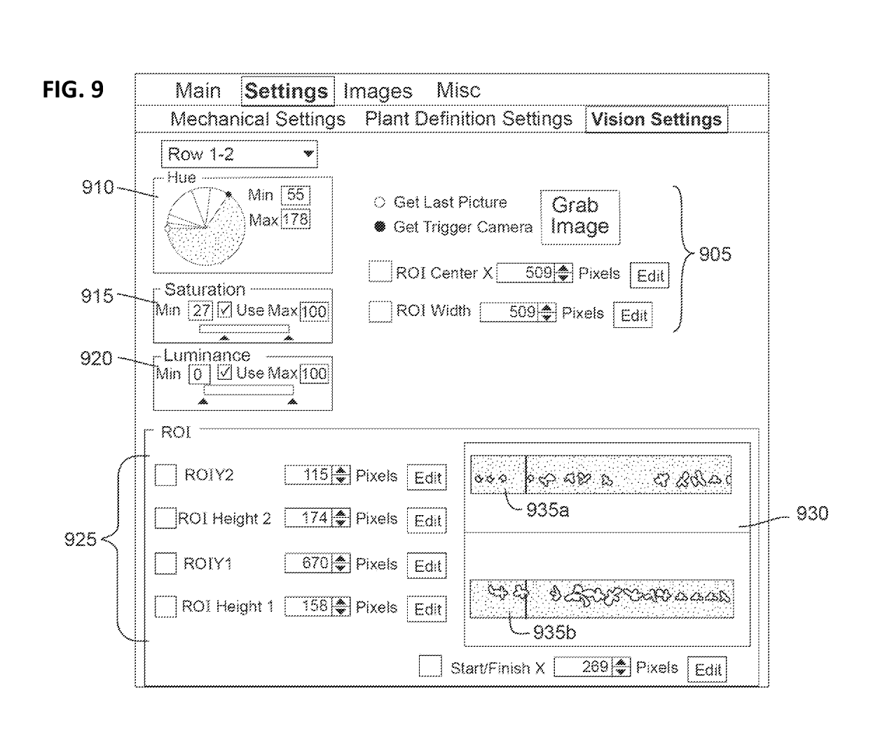

Using the machine-vision system described in Example 1, digital images of a crop bed of lettuce seedlings in Yuma, AZ were captured and a region of interest (ROI) within the image was analyzed. FIGS. 9 and 10 depict user-interface screen shots of an exemplary identification and analysis of plants in the ROI.

During operation, the camera is triggered to capture images at regular distance intervals as the camera is moved along the plant row in the direction of travel of a tractor. The distance used is determined from the procedures for calibrating image pixel size described above. Once the physical dimensions of a pixel are known, the dimensions of the image captured can be calculated from the number of pixels along the image length and width.

The camera used herein captures images having a length of 1360 pixels, and a width of 1024 pixels. Pixel dimension values were determined from two captured images (Lanes). The average of the calculated values of pixel dimension for Lane 1 (42.518 pixels/inch) and Lane 2 (42.923 pixels/inch) was 42.721 pixels/inch. As a result, the captured image had a real-world length of 31.8 inches and width of 24.0 inches. Based on this size, the program calculates the recommended distance needed between pictures for each lane captured. The average of the two values is calculated and entered into the user editable “Avg Inches between Pics” box in the user interface, which is displayed when an “Average” button is pressed. This value, which is entered and stored in memory, is converted from inches to a corresponding number of encoder pulses based on the encoder wheel calibration value also stored in memory. In this example, this encoder measurement value was 28.75 pulses/inch. Therefore, a calculated average distance between pictures of 21.673 inches is converted to 623 encoder pulses.

As the machine moves, the control system sends a signal to trigger the camera every 623 encoder pulses, and this image and the encoder count value when the signal was sent are stored in memory. The camera is triggered at distance intervals that are smaller than the length of the captured image length. Sequential images are spliced together with overlapping portions aligned to form a new composite-view image comprised of two sequential images. This composite image is the image that is analyzed and used for determining the locations of plant part and non-plant part locations. This procedure allows for selective treatment decisions to be made continuously and accurately as the machine travels down a crop row. More than two sequential images could be spliced together using the procedures developed if desired. The camera trigger distance dictates the minimum distance the camera must be positioned from the target location of the implement (treatments means). This distance is based on the field of view the camera “sees” and is therefore dependent on camera height if the camera is pointed in a downward direction.

Captured images of the crop row were analyzed to determine the existence and location of plant and non-plant pixels. FIG. 9 depicts a user-interface screen through which a user can adjust the analysis as desired, such as input fields to adjust the overall size of the image captured for analysis. The upper left corner of the figure shows the minimum and maximum H 910, S 915, and L 920 threshold values used to determine whether a pixel is a part of a plant or not. As shown in the figure, each HSL threshold value is adjustable using the user interface. In this example, the minimum and maximum threshold values for H were set to 55 and 178; minimum and maximum threshold values for S were set to 27 and 100; and minimum and maximum threshold values for L were set to 0 and 100, respectively.

Following image capture, each image is divided into one or more ROIs for analysis. FIG. 9 illustrates the user interface for changing the ROI parameters. The lower right corner shows the analyzed image 930 of a nominally 22-inch wide, raised bed that was top planted using two rows of iceberg lettuce plants. Displayed within each row image is a rectangle surrounding crop plants that defines the ROIs to be analyzed 935a and 935b. The locations of these rectangle boundaries can be individually user-adjusted using the shown fill-in boxes 925 and/or made larger or smaller using a computer mouse or other suitable data input device. Defining the size and location of the ROI ensures that the plants of interest are detected reliably should the machine drift laterally, and minimizes the amount of processing time for image-analysis. Processing time is directly proportional to image size, and is a limiting factor in machine vision systems.

In the ROI analyzed under the parameters shown in FIG. 9, an exemplary pixel representing part of a crop plant had RGB values of 123, 149 and 85, respectively, which were converted to HSL values of 84, 58 and 42, respectively. All of these values satisfied the conditions to be designated a plant pixel. Conversely, an exemplary pixel determined not to be representative of a crop plant had HSL values (after conversion from RGB) of 53, 42 and 16, respectively. Because the H value of 53 is less than the minimum threshold value of 55, the pixel was identified as not being a plant part.

A zoomed-in view of the analyzed ROI 1005 is depicted in FIG. 10. Plant pixels are shown as unbroken white space 1010. Non-plant pixels are shown as the speckled background 1015. A non-analyzed representation 1020 of the ROI is also shown on either side of the analyzed ROI 1005. FIG. 10 (bottom) also depicts the plant pixel distribution plot 1025 that the controller generated for the same analyzed ROI. For this distribution, the number of pixels in each column across all rows within the ROI was plotted versus distance in terms of column width (in this example, one pixel, which is about 0.024 inches wide).

As described herein, a plant in any given ROI is identified when more than a predefined number of adjacent columns (“minimum plant length”) each contain at least one white pixel more than a predefined number of white pixels (“noise” value). In particular examples, “minimum plant length” and “noise” are pre-set or standard values. For the analysis shown in FIG. 10, minimum plant length was set to 0.25 inches (11 pixels), a noise level set to 5 pixels, and an array filter size set to 10 pixels.

Once a plant is identified, the left-most column in the array of adjacent columns is taken to be the trailing-edge location while the right-most column is taken to be the leading-edge location. This calculation of the precise plant-boundary locations is used by the controller to correlate selective action with a particular location.

### Example 6

### Selective Thinning of Lettuce Seedlings with Spray-Treatment Means

The previous example describes identification of plants and plant boundaries in a ROI with pixel level accuracy. This example describes the manner in which this information is used by the system described in Example 1 to take selective action in the ROI, such as selective thinning of lettuce seedlings.

As described above, one or more treatment means can be mounted to the support. Operation of each treatment means is computer-controlled through output of electronic signals from the controller. The exemplary system depicted in FIG. 2 has four different treatment means: two spray assemblies 260a and 260b, and two different blade types 265 and 270. Control of treatment means is based on relative distance. Here, the relative distance used for treatment control is the distance from the camera center to each treatment means. Values of relative distance are user-adjustable and entered into the controller through the user interface, for example on a screen including boxes labeled “Inches to Terminator 1,” “Inches to Terminator 2,” and “Inches to Terminator 3.” Adjustable distances allow the user to shift the real-world location in which the treatment means are actually applied and to account for errors caused by the time required for actuators to process signals and the time required to physically move a treatment means to the target location. Similarly, the particular treatment means to be used by the system can be user-selected or is a pre-set default.

Target location and duration of a given treatment are calculated from a combination of plant-location data (obtained by analysis of the ROI), and user-inputted settings. FIG. 11 depicts a user-interface screen shot in which a user can modify optimum plant spacing, minimum plant spacing, leading-edge buffer distance, and trailing-edge buffer distance, filter size for minimum plant length array, and noise levels. In this screen shot, the programmed settings used were: minimum plant length of 0.25 inches (11 pixels), noise level of 5 pixels, array filter size of 10 pixels, a 1-inch buffer on both the leading and trailing edges of the plant to be kept, an optimum plant spacing of 9 inches, and a minimum plant spacing of 6 inches.

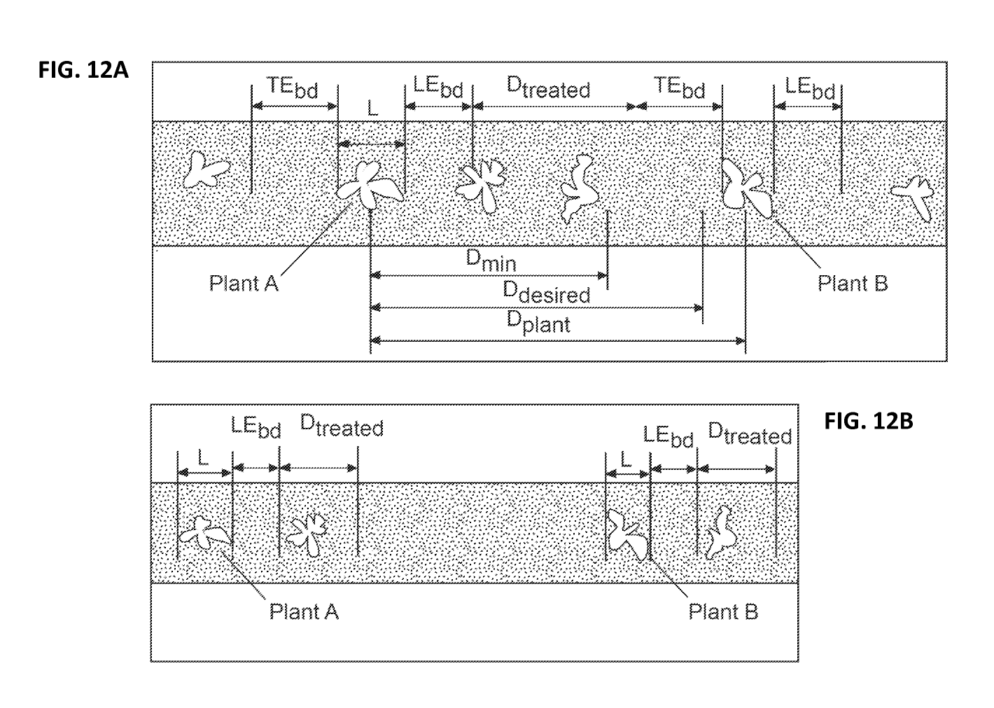

The application of the above settings to an analyzed ROI is depicted in FIG. 12A. As illustrated in the figure, “Plant A” was identified as a crop plant. It has a length “L” calculated from determined trailing-edge and leading-edge locations. The horizontal location of the trailing buffer zone is computed from the trailing-edge location and a user-inputted “Trailing Edge Buffer Distance” (TE_{bd}), and stored in memory. Similarly, the horizontal location of the leading buffer zone is computed from the leading-edge location and a user-inputted “Leading Edge Buffer Distance” (LE_{bd}), and stored in memory. The program then searches the analyzed ROI to identify the next plant to be “saved” based on TE_{bd}, LE_{bd}, desired plant spacing (D_{desired}) and minimum plant spacing distances (Dₘᵢₙ). The program identifies the next plant to be saved as the one whose center is located at a distance from the “already saved” plant that is greater than the Dₘᵢₙ and at a distance that is closest to D_{desired}. “Plant B” as the next plant to be saved.

As discussed above, the systems disclosed herein can be programmed to identify and operate in crop stands regardless of planting configuration. For example, the controller can be programmed to recognize and operate in crop stands that are sown according to “two-drop” or “three-drop” planting methods. In such methods, two or three seeds are sown one to two inches apart in groups. These groups are nominally sown at the desired final plant spacing, typically ten to twelve inches apart. For two-drop and three-drop crop stands, the controller can be programmed to terminate plants for a preset distance after the first plant in the group is identified in a ROI. As illustrated in FIG. 12B, once a first plant (Plant A) has been identified in a two-drop planting scheme and selected as a saved plant, its plant edge locations are determined and treated distances are established as described above.

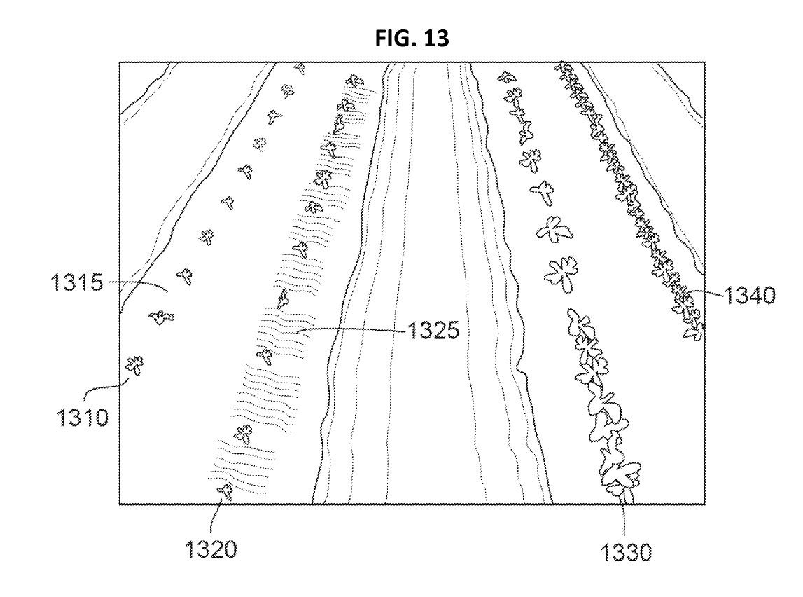

Typical performance results using the pressurized spray-based treatment means are depicted in FIG. 13. The machine-vision system was operated in thinning mode with desired plant spacing set to 7 inches, trailing-buffer-edge and leading-buffer-edge distances set to 1.5 inches, minimum plant-spacing distance set to 4 inches, minimum plant length set to 0.25 inches, distance to selected spray-treatment means set to 56.75 inches, noise-level set to 5 pixels, array-filter size set to 10, minimum and maximum threshold values for H set to 55 and 178 respectively, minimum and maximum threshold values for S set to 27 and 100 respectively, and minimum and maximum threshold values for L set to 0 and 100 respectively. Encoder and pixel-calibration settings were 28.75 pulses/inch and 42.68 pixels/inch respectively, and the camera was set to capture and image every 21.673 inches. Travel speed was 1 mph. The machine-vision system was tested in a field planted with head lettuce nominally planted as 2-inch spacing. The machine was used when lettuce seedlings were at the 2-leaf to 3-leaf stage of growth, approximately half an inch in diameter.

The treatment solution was a spray solution of 10% sulfuric acid mixed with SIGNAL™ Spray Colorant (Precision Laboratories Inc., Waukegan, Ill.) and polyacrylamide anti-drift product at a concentration of 4 ounces per 100 gallons of treatment solution.

FIG. 13 illustrates the results of the trial one day after spray treatment. Here, the far left crop row 1310 was thinned using the machine vision system; the right row 1320 on the same bed top was thinned by hand. Plants in the far right rows 1330 and 1340 were not thinned.

One goal of the trial depicted in FIG. 13 was to identify differences in plant growth between hand-thinned and machine-thinned lettuce. Fourteen days after treatment, no discernable visible differences in plant growth or health were noticed between the rows thinned by the two thinning methods 1310 and 1320. As shown in FIG. 13, the only major difference between the two thinning methods was that soil 1315 between the plants in the machine-thinned row 1310 was not disturbed. In contrast, the soil 1325 between the plants in the hand thinned row 1320 was visibly disturbed. Decreased soil disturbance is advantageous, in that few new weed seeds are brought to the soil surface, and pits in the soil are not formed. Minimizing soil pits is also advantageous because soil pits store water, which can promote certain plant diseases when in contact with plant tissue.

In another trial of the machine-vision system, a mechanical treatment means composed of a narrow blade was used to undercut the roots of unwanted lettuce seedlings. The blade was configured to be dragged through the soil and raised in the trailing-edge and leading-edge buffer regions. The blade is lowered again after it passes the next saved plant by a distance equivalent to the preset leading-edge buffer distance. Plant thinning with this system also induced minimal soil disturbance as compared to traditional thinning with handheld hoes and yielded good results for thinning lettuce plants nominally planted 2 inches apart.

A second mechanical treatment means, using a linear actuated blade was also tested. Similar to the narrow blade, the linear blade thins unwanted seedlings by undercutting plant roots below the soil surface. The linear actuated blade also effectively thinned lettuce seedlings.

### Example 7

### Comparison of Automated and Hand Plant Thinning

This example compares the results of plant thinning with several treatment means to traditional hand thinning. The machine vision system used in this example is as described in Example 1, except the spray nozzles were enclosed within a “hooded” box assembly to protect sprayed treatment from wind effects.

Presented in Table I, below, are data comparing the performance of the machine-vision system as described above to hand thinning. Lettuce seedlings were thinned either using the machine vision system with the indicated treatments. The far left column lists the seven treatments tested, including hand thinning (control) and six treatments used by the automated thinning machine. Of these six, the automated thinning machine was evaluated spraying five different liquid products (two acids, two fertilizers, and one herbicide) known to kill plants and one mechanical method (knife blade—“hula hoe” design). The second column is the cost of the material sprayed for the flow rates and travel speeds used. The third column is the average of the measured distance to each live plant after thinning. The fourth column is the number of live plants per acre after thinning. The fifth column is the time required by a hand laborer to walk through the field with a hand hoe and remove weeds in the row between crop plants and any lettuce plants missed during the thinning operation. The data show that there was no difference in machine performance as compared to hand thinning when the liquid products sulfuric acid and paraquat were used. The data also show that both sulfuric acid and paraquat provided faster and generally more cost effective treatment means than any other treatment using the machine vision system.

TABLE 1
Comparison of Treatment Means

| | Material | Plant | Plant | |
|---|---|---|---|---|
| | Cost | Spacing | Count | Time |
| Treatments | per acre | inch | per/acre | hr/acre |
| Hand | NA | 9.5ab* | 28,936b | 2.8d |
| Sulfuric Acid (10%) | $4.30 | 10.2a | 26,758b | 2.7d |
| Paraquat | $0.35 | 9.6ab | 27,692b | 2.8d |
| Acetic Acid (20%) | $32.27 | 8.9b | 30,181b | 3.0cd |
| AN20 | $20.00 | 7.5c | 36,715a | 3.3bc |
| UAN 32 | $23.09 | 7.3c | 37,337a | 3.3b |
| Knife Blade | NA | 6.8c | 41,071a | 3.7a |

*Within columns, means followed by the same letter are not significantly different by Duncan's new multiple range test (P = 0.05).

### Example 8

### Use of Multiple Spraying Assemblies

This example describes use of multiple spraying assemblies in conjunction with the machine vision system described in Example 1.

Trials utilizing two spraying assemblies attached to the system described in Example 1 proved the feasibility of using multiple spraying assemblies to simultaneously kill unwanted plants and benefit saved plants. In this trial, a 10% concentration of sulfuric acid was used to thin lettuce seedlings. A second spray assembly was used to simultaneously spray a molar equivalent basic solution of sodium bicarbonate to neutralize any acid that drifted onto the saved plants. The trial was conducted at 1 mph.

### Example 9

### Use of a Hooded Spray Assembly

This example describes use of a hooded spray assembly in conjunction with the machine-vision system described herein.

Many agricultural pesticides have restricted-use labels and can only be used in hooded sprayers after the crop has emerged. Thus, one of skill will appreciate that mounting one or more spray assemblies in a hooded sprayer will expand the range of spray treatments available for use with the systems described herein. In such a mounted assembly, the spray nozzle assembly can be mounted within the same box as the imaging system camera. This box would be fabricated out of light-weight, corrosive-resistant materials such as sheets of polyethylene plastic with structural support provided by lengths of “L”-shaped stainless steel. The box would be mounted on wheels positioned close to the seed row and attached to a main machine frame by arms that allow the box to pivot and “float” relative to the machine frame. The floating design keeps the machine-vision system camera and spray nozzles positioned at a constant height above the ground surface.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

1. An agricultural system, comprising:
a support movable in a trajectory along an array of plants;
an image sensor comprising a camera, mounted to the support, the image sensor camera producing real-time images on an electronic image-capture device containing an array of pixels;
a distance-measuring device that produces, in real time, data regarding position of the support in the trajectory relative to a positional reference;
a first controller connected to the image sensor and to the distance-measuring device, the first controller being programmed or otherwise configured (a) from the data obtained from the distance-measuring device and at each of selected discrete distances in the trajectory from the reference, to generate an activate signal triggering the image sensor to obtain an image of a respective region of interest (ROI) of the array situated at the respective selected distance from the reference, (b) to receive pixelated image data of the ROI image from the image sensor, (c) at pixels of the image, determine whether light received at the pixels is indicative of plant versus non-plant, (d) to determine a data distribution of plant-indicating pixels and non-plant-indicating pixels as a function of distance in the ROI and hence relative to the reference, and (e) in the distribution, determine respective positions of leading and trailing edges of plant-indicating pixels and correlating these positions with desired action or non-action to be taken with respect to selected plants in the ROI.

2. The system of claim 1, further comprising a user interface for programming the controller and displaying data of the plant-indicating and non-plant-indicating pixels in the ROI.

3. The system of claim 1, wherein the first controller is further programmed to produce, with respect to a plant in the ROI determined to be at a position correlated with action, at least one implement-actuation command to take at least one desired action on, or relative to the plant.

4. The system of claim 1, further comprising a second controller, wherein the second controller is programmed to produce, with respect to a plant in the ROI determined to be at a position correlated with action, at least one implement-actuation command to take at least one desired action on, or relative to the plant, the second controller being connected to and programmable using the user interface.

5. The system of claim 1, further comprising at least one implement connected to the first controller, wherein the implement receives the actuation command and takes the desired action.

6. The system of claim 4, further comprising at least one implement connected to the second controller, wherein the implement receives the actuation command and takes the desired action.

7. The system of claim 3, comprising multiple implements, wherein each of the multiple implements receives a respective implement-actuation command in response to which each implement takes the desired action.

8. The system of claim 5, wherein the implement or implements are configured, upon receiving the actuation command, to manipulate a plant or region of soil associated with the plant.

9. The system of claim 5, wherein at least one implement comprises a spray nozzle.

10. The system of claim 7, wherein each of the multiple implements comprises a respective spray nozzle.

11. The system of claim 5, wherein at least one implement comprises a blade.

12. The system of claim 2, wherein the user interface is connectable for use only when needed.

13. The system of claim 1, wherein the support is pulled or pushed along the trajectory.

14. The system of claim 1, wherein the support moves itself along the trajectory.

15. The system of claim 1, wherein the distance-measuring device is a rotary or linear encoder.

16. An agricultural system, comprising:
support means movable in a trajectory along an array of plants;
means, coupled to the support means, comprising a camera, for producing pixelated electronic images of respective portions of the array, each image consisting of an array of pixels;
means for measuring distance of the support means in the trajectory relative to a positional reference;
means for actuating the camera to take respective images of respective regions of interest (ROIs) of the plant array along the trajectory at respective selected distances from the reference;
means for determining, in each image, whether light received at each pixel thereof is indicative of plant versus non-plant; and
means for determining, in each image, respective positions of leading and trailing edges of plant-indicating pixels and for correlating these positions, with desired action or non-action to be taken with respect to selected plants in the ROI.

17. The system of claim 16, further comprising:
implement means mounted to said support means; and
means for actuating the implement means to take action with respect to a plant in the ROI determined to be at a position correlated with the action.

18. A method for manipulating plants in situ, comprising:
while moving at least one implement in a trajectory along an array of plants, determining the position of the implement in real time relative to a positional reference;
while moving the implement, obtaining in real time, a series of pixelated images of respective portions of the array located in respective regions of interest (ROI) situated at discrete respective distances from the reference;
in each pixelated image, determining whether respective image light received at each of the pixels is indicative of plant versus non-plant;
in each pixelated image, determining respective leading and trailing edges of plant-indicating pixels and correlating these positions, with desired action or nonaction to be taken with respect to selected plants in the respective ROI; and
actuating the implement to take action with respect to a plant in the ROI determined to be at a position correlated with the action.

19. The method of claim 18, wherein correlating the respective leading and trailing edges of plant-indicating pixels with desired non-action comprises selective thinning of the array of plants, comprising:
identifying the plant-indicating pixels as plants within a desired plant size;
identifying which plants to keep among the plants of desired plant size;
correlating the locations of the plants to keep with non-action of the implement; and switching off the implement actuation command at the location of plants to keep, thereby selectively thinning the array of plants.

20. The method of claim 18, wherein at least one implement comprises a nozzle.

21. The method of claim 18, wherein at least one implement comprises a blade.

22. The method of claim 18, wherein the action is plant thinning, weeding, spot spraying, watering, or fertilizing.

23. The method of claim 18, further comprising at least one additional action with respect to the plant in the ROI.

24. The method of claim 23, wherein the additional action comprises plant thinning, weeding, spot spraying, watering, or fertilizing.
